(19) United States
(12) Patent Application Publication
(10) Pub. No.: US 2021/0386074 A1
(43) Pub. Date: Dec. 16, 2021

(54) Plant Extract Compositions and Methods of Making and Using the Same

(71) Applicant: Greenology Products, Inc., Raleigh, NC (US)

(72) Inventors: Adam McCarthy, Raleigh, NC (US); Lara Samofal Madding, Cary, NC (US); Liana Victoria Palaikis, Raleigh, NC (US); Marilyn Hull Boothe, Raleigh, NC (US)

(21) Appl. No.: 17/389,651

(22) Filed: Jul. 30, 2021

Related U.S. Application Data
(63) Continuation-in-part of application No. PCT/US2020/031697, filed on May 6, 2020.
(60) Provisional application No. 62/843,870, filed on May 6, 2019.

(51) Int. Cl. A01N 65/08; A01N 59/00; A01N 25/02

(52) U.S. Cl. CPC A01N 65/08; A01N 25/02; A01N 59/00

(57) ABSTRACT

Plant infusions or extracts and compositions including the same are disclosed. The plant infusions or extracts and compositions including the plant infusions or extracts include ingredients that may comply with the United States Department of Agriculture's (USDA's) National Organic Program (NOP) for products labeled as “organic” or “made with organic” and can be used as mitigating compositions and/or a preservative.

### CROSS-REFERENCE TO RELATED APPLICATIONS

The present application is a bypass continuation-in-part application of PCT International Application No. PCT/US2020/031697, filed May 6, 2020, which claims priority from and the benefit of U.S. Patent Application No. 62/843,870, filed May 6, 2019, the disclosures of each of which are hereby incorporated herein by reference in their entireties.

### FIELD

The presently disclosed subject matter relates to extracts, infusions, and extract and/or infusion compositions. These infusion and infusion compositions can be used as mitigating compositions and/or as preservatives. In particular, the infusions are derived from plants and the infusion compositions are derived from plant infusions. Both can be used to mitigate against pathogenic and non-pathogenic organisms and/or to preserve food, beverages, pharmaceuticals, nutraceuticals, cosmetics, personal care products, cleaning compositions and the like.

### BACKGROUND

Preservatives and/or mitigating agents are generally substances that are added to a product to inhibit or prevent alterations to the substance as a result of microbial growth. Thus, such compositions prevent or significantly minimize contamination by pathogenic and non-pathogenic microorganisms.

One of the most limiting issues with natural and certified organic products is that standard market preservatives are generally unacceptable in natural or organic formulations. As a result, natural and organic products are vulnerable to many types of contamination. Accordingly, there is a need for naturally derived and minimally processed compositions that can be classified as natural, organic or certified organic and impart effective preservation and/or mitigating activity to the desired product.

### SUMMARY

The presently disclosed subject matter provides compositions for use as a preservative and/or mitigating composition generally. In particular embodiments, the presently disclosed subject matter provides a composition including a mixture of at least one infusion or extract, such as a plant infusion or extract, and hydrogen peroxide, and optionally at least one stabilizing agent, such as glycerin.

In further embodiments, the presently disclosed subject matter provides a composition including a polyphenol and/or phenolic acid (and/or other phenolic compounds), and hydrogen peroxide, and optionally at least one stabilizing agent, such as glycerin.

Embodiments of the presently disclosed subject matter further provide a plant infusion or extract including: (a) a solvent selected from the group consisting of water, alcohol, chloroform, acetone, petroleum ether, and ionic liquid and combinations thereof; (b) hydrogen peroxide; and (c) a plant material.

According to further embodiments, the presently disclosed subject matter provides a product including a composition including a polyphenol and/or phenolic compound and hydrogen peroxide, and optionally at least one stabilizing agent, such as glycerin, or including a plant infusion or extract described herein.

Embodiments of the presently disclosed subject matter further provide a method of making a plant infusion or extract, the method includes adding a plant material to a solvent; filtering the solution; and adding hydrogen peroxide to the filtered solution to obtain a plant infusion or extract composition.

The compositions and methods described herein can be provided in the form of natural, organic or certified organic compositions and methods.

Certain aspects of the presently disclosed subject matter have been stated hereinabove. Other aspects will become evident as the description proceeds when taken in connection with the accompanying examples presented herein below.

### BRIEF DESCRIPTION OF THE DRAWINGS

FIG. 1 presents a flow chart illustrating the manufacture of a honeysuckle extract composition according to embodiments of the present invention.

### DETAILED DESCRIPTION

The presently disclosed subject matter now will be described more fully hereinafter with reference to the accompanying examples, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated examples. Therefore, it is to be understood that the presently disclosed subject matter is not to be limited to the specific embodiments disclosed and that modifications and other embodiments are intended to be included within the scope of the appended claims.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items. Moreover, it should be noted that various embodiments of the presently disclosed compositions of matter may “comprise,” “consist” or “consist essentially of” the components or ingredients disclosed herein.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of any numerical ranges by endpoints or in succession includes all numbers, e.g., whole integers, including fractions thereof and decimals, subsumed within that range or sequence (for example, the recitation of 1 to 5 includes 1, 2, 3, 4, and 5, as well as fractions thereof, e.g., 1.5, 2.25, 3.75, 4.1, and the like and any range within that range and at least 95%, 96%, 97%, 98% or 99% includes all decimals in between and up to the next higher percentage up to 100).

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents form part of the common general knowledge in the art.

As used herein, the term “plant” is used in a generic sense, and includes, for example, woody-stemmed plants such as trees and shrubs, herbs, vegetables, fruits, agricultural crops, and ornamental plants and flowers and further includes whole plants and any portions thereof.

Vegetables include, but are not limited to, leafy green vegetables such as lettuce (e.g., Lactuea sativa), spinach (Spinaca oleracea), and cabbage (Brassica oleracea), various roots, such as potatoes (Solanum tuberosum) and carrots (Daucus), bulbs, such as onions (A. Ilium sp.), herbs, such as basil (Ocimum basilicum), oregano (Origanum vulgare), dill (Anethum graveolens), as well as soybean (Glycine max), lima beans (Phaseolus limensis), peas (Lathyrus spp.), corn (Rea mays), broccoli (Brassica oleracea italica), cauliflower (Brassica oleracea bottytis), and asparagus (Asparagus officinalis).

Fruits include, but are not limited to, tomatoes (Lycopersicon esculentum), apples (Malus domestica), bananas (Musa acuminata, Musa balbisiana, and Musa×paradisiaca), pears (Pyrus communis), papaya (Carica papaya), mangoes (Mangifera indica), peaches (Prunus persica), apricots (Prunus armeniaca), nectarines (Prunus persica nectarina), oranges (Citrus sp.), lemons ((Citrus limonia), limes (Citrus aurantifolia), grapefruit (Citrus paradise), tangerines (Citrus noblis deliciosa), kiwi (Actinidia chinenus), melons such as cantaloupe (C. cantalupensis) and musk melon (C. melo), pineapple (Aranas comosus), persimmon (Diospyros sp.), various small fruits including berries such as strawberries (Fragaria), blueberries (Vaccinium sp.) and raspberries (e.g., Rubus ursinus), green beans (Phaseolus vulgaris), members of the genus Cucumis such as cucumber (C. sativus), and avocados (Persea americana).

Ornamental and edible plants and flowers include, but are not limited to, azalea (Rhododendron spp.), hydrangea (Macrophylla hydrangea), hybiscus (Hibiscus rosasanensis), snapdragons (Antirrhinum sp.), poinsettia (Euphorbia pulcherima), cactus (e.g. Cactaceae schlumbergera truncata), begonias (Begonia sp.), roses (Rosa spp.), tulips (Tulipa sp.), daffodils (Narcissus spp.), dandelions (Taraxacum offinale), petunias (Petunia hybrida), carnation (Dianthus catyophyllus), lily (e.g., Lilium sp.), gladiolus (Gladiolus sp.), alstroemeria (Alstoemeria brasiliensis), anemone (e.g., Anemone blanda), columbine (Aquilegia sp.), aralia (e.g., Aralia chinensis), aster (e.g., Atser carolinianus), bougainvillea (Bougainvillea sp.), camellia (Camellia sp.), bellflower (Campanula sp.), cockscomb (celosia sp.), falsecypress (Chamaecyparis sp.), chrysanthemum (Chrysanthemum sp.), clematis (Clematis sp.), cyclamen (Cyclamen sp.), freesia (e.g., Freesia refracta), wisteria (Wisteria sp.), orchids of the family Orchidaceae, artichoke (e.g., Cynara cardunculus), honeysuckle of the genus Lonicera and species Lonicera caprifolium (goat-leaf honeysuckle, perfoliate honeysuckle), Lonicera periclymenum (European honeysuckle or woodbine), Lonicera japonica (Japanese honeysuckle, white honeysuckle, or Chinese honeysuckle), Lonicera sempervirens (coral honeysuckle or trumpet honeysuckle), Lonicera hildebrandiana (Burmese honeysuckle), Lonicera tartarica (Tartarian honeysuckle), Lonicera fragrantissima (winter honeysuckle), Lonicera pileate (privet honeysuckle), Lonicera nitida (box honeysuckle) and Lonicera syringantha (lilac-flowered honeysuckle).

In particular embodiments, the plant includes sweet potatoes, eggplant, green beans, black beans, white beans, red beans, kidney beans, pinto beans, green peas, black-eyed peas, chick peas, carrots, tobacco, chili peppers, grapes, plums, prunes, raisins, black currants, cherries, apples, lemons, limes, oranges, kiwi, pears, tomatoes, blueberries, black berries, strawberries, raspberries, cranberries, coffee beans, hazel nuts and pecans.

As used herein, “extract” refers to a preparation including mainly an active ingredient(s) of a substance. “Extract” and “infusion” are used interchangeably herein where the active ingredient(s) may be obtained through processes including, steeping, soaking, boiling, separating, solubilizing, suspending, straining and/or other processes known to those skilled in the art. The active ingredient(s) may be in a concentrated form and may be dry or liquid. The active ingredient(s) may also be pure, artificial or synthetic. The extract may be a pure extract or an artificial or synthetic extract and may be dry or liquid. In certain embodiments, the “purity” of any given agent in a composition may be specifically defined. For instance, certain compositions may include, for example, an agent that is at least 80, 85, 90, 91, 92, 93, 94, 95, 96, 97, 98, 99, or 100% pure, including all decimals in between, as measured, for example and by no means limiting, by high pressure liquid chromatography (HPLC), a well-known form of column chromatography used frequently in biochemistry and analytical chemistry to separate, identify, and quantify compounds.

As used herein, the term “natural” refers to the state of being obtained from a natural source (plant or non-plant source, e.g., animal or insect source) in a substantially unmodified form, namely in the form which exists in the material natural origin, or a composition including components that are obtained in the aforementioned manner. Natural components may be obtained from their natural source by way of extraction or other ways of separation. “Natural” is also intended to encompass green, sustainable, holistic, bio-degradable, nature-identical, homeopathic, eco-friendly, earth-friendly, environmentally safe, paraben-free and/or non-toxic.

As used herein, the term “organic” refers to a product or ingredient that complies with 7 CFR § 205.301, which establishes organic content requirements for labeling and marketing purposes. Four categories of organic content have been established by the United States Department of Agriculture (USDA) under the National Organic Program (NOP): “100 percent organic,” which is defined as products comprising 100 percent organic ingredients; “organic,” which is defined as products comprising 95 percent or more organic ingredients; “made with organic,” which is defined as products comprising between 70 percent and 95 percent organic ingredients; and “ingredient panel only,” which is defined as products comprising less than 70 percent organic ingredients.

More particularly, under 7 CFR § 205.301, all of the ingredients in products sold, labeled, or represented as “100 percent organic” must be certified organic and contain (by weight or fluid volume) 100 percent organically produced ingredients.

Products sold, labeled, or represented as “organic” must contain (by weight or fluid volume) not less than 95 percent organically produced raw or processed agricultural products. Any remaining product ingredients must be organically produced, unless not commercially available in organic form, or must be nonagricultural substances or non-organically produced agricultural products as specified on the National List under 7 CFR § 205.605. Accordingly, non-organic ingredients allowed per National List may be used, up to a combined total of five percent of non-organic content.

Products sold, labeled, or represented as “made with organic” must contain (by weight or fluid volume) at least 70 percent organically produced ingredients. Accordingly, multi-ingredient agricultural products in the “made with organic” category must include at least 70 percent certified organic ingredients. Although any remaining agricultural products are not required to be organically produced, non-agricultural products must be specifically allowed on the National List under 7 CFR § 205.605.

Multi-ingredient products having less than 70 percent certified organic content are not qualified to be certified. Accordingly, any non-certified product must not include the USDA organic seal anywhere or the word “organic” on a principal display panel. Such products may only list certified organic ingredients as organic in the ingredient list and the percentage of organic ingredients. Remaining ingredients are not required to follow the USDA organic regulations.

Further, under 7 CFR § 205.605, specified nonagricultural (nonorganic) substances may be used as ingredients in or on processed products labeled as “organic” or “made with organic,” including the following nonsynthetic and synthetic ingredients:

Nonsynthetics: acids (alginic; citric—produced by microbial fermentation of carbohydrate substances; and lactic); agar-agar; animal enzymes—(rennet—animals derived; catalase—bovine liver; animal lipase; pancreatin; pepsin; and trypsin); bentonite; calcium carbonate; calcium chloride; calcium sulfate—mined; carrageenan; dairy cultures; diatomaceous earth—food filtering aid only; egg white lysozyme (CAS #9001-63-2); enzymes derived from edible, nontoxic plants, nonpathogenic fungi, or nonpathogenic bacteria; flavors, nonsynthetic sources only and must not be produced using synthetic solvents and carrier systems or any artificial preservative; gellan gum (CAS #71010-52-1)—high-acyl form only; glucono delta-lactone—production by the oxidation of D-glucose with bromine water is prohibited; kaolin; L-malic acid (CAS #97-67-6); magnesium sulfate, nonsynthetic sources only; microorganisms—any food grade bacteria, fungi, and other microorganism; nitrogen—oil-free grades; oxygen—oil-free grades; perlite—for use only as a filter aid in food processing; potassium chloride; potassium iodide; sodium bicarbonate; sodium carbonate; tartaric acid—made from grape wine; waxes—nonsynthetic (carnauba wax; and wood resin); yeast—nonsynthetic, growth on petrochemical substrate and sulfite waste liquor is prohibited (autolysate; bakers; brewers; nutritional; and smoked—nonsynthetic smoke flavoring process must be documented); and synthetics: activated charcoal (CAS #s 7440-44-0; 64365-11-3)—only from vegetative sources; for use only as a filtering aid; alginates; ammonium bicarbonate—for use only as a leavening agent; ammonium carbonate—for use only as a leavening agent; ascorbic acid; calcium citrate; calcium hydroxide; calcium phosphates (monobasic, dibasic, and tribasic); carbon dioxide; cellulose—for use in regenerative casings, as an anti-caking agent (non-chlorine bleached) and filtering aid; chlorine materials—disinfecting and sanitizing food contact surfaces, except that residual chlorine levels in the water shall not exceed the maximum residual disinfectant limit under the Safe Drinking Water Act (calcium hypochlorite; chlorine dioxide; and sodium hypochlorite); cyclohexylamine (CAS #108-91-8)—for use only as a boiler water additive for packaging sterilization; diethylaminoethanol (CAS #100-37-8)—for use only as a boiler water additive for packaging sterilization; ethylene—allowed for postharvest ripening of tropical fruit and degreening of citrus; ferrous sulfate—for iron enrichment or fortification of foods when required by regulation or recommended (independent organization); glycerides (mono and di) for use only in drum drying of food; glycering—produced by hydrolysis of fats and oils; hydrogen peroxide; lecithing—bleached; magnesium carbonate—for use only in agricultural products labeled “made with organic” prohibited in agricultural products labeled “organic”; magnesium chloride—derived from sea water; magnesium stearate—for use only in agricultural products labeled “made with organic” prohibited in agricultural products labeled “organic”; nutrient vitamins and minerals, in accordance with 21 CFR 104; 20, Nutritional Quality Guidelines For Foods; octadecylamine (CAS #124-30-1)—for use only as a boiler water additive for packaging sterilization; ozone; pectin (low-methoxy); peracetic acid/peroxyacetic acid (CAS #79-21-0)—for use in wash and/or rinse water according to FDA limitations; for use as a sanitizer on food contact surfaces; phosphoric acid—cleaning of food-contact surfaces and equipment only; potassium acid tartrate; potassium carbonate; potassium citrate; potassium hydroxide—prohibited for use in lye peeling of fruits and vegetables except when used for peeling peaches during the Individually Quick Frozen (IQF) production process; potassium iodide—for use only in agricultural products labeled “made with organic,” prohibited in agricultural products labeled “organic”; potassium phosphate—for use only in agricultural products labeled “made with organic,” prohibited in agricultural products labeled “organic”; silicon dioxide; sodium acid pyrophosphate (CAS #7758-16-9)—for use only as a leavening agent; sodium citrate; sodium hydroxide—prohibited for use in lye peeling of fruits and vegetables; sodium phosphates—for use only in dairy foods; sulfur dioxide—for use only in wine labeled “made with organic grapes,” provided that total sulfite concentration does not exceed 100 ppm; tartaric acid—made from malic acid; tetrasodium pyrophosphate (CAS #7722-88-5)—for use only in meat analog products; tocopherols—derived from vegetable oil when rosemary extracts are not a suitable alternative; and xanthan gum.

The presently disclosed subject matter provides compositions for use as a preservative and/or mitigating agent and/or composition generally. The compositions described herein can be provided in the form of a natural, organic or certified organic composition. As such, the compositions can provide effective preservative and/or mitigating activity, are generally regarded as safe (or safer) to use and generally exhibit no greater toxicity (or are less toxic) to the user and the environment than existing preservative and/or mitigating compositions used in the same capacity while also complying with the requirements of the United States Department of Agriculture's (USDA's) National Organic Program (NOP) for products labeled as “organic” or “made with organic.”

Embodiments of the present inventive concept include a composition comprising a mixture of at least one infusion or extract, such as plant infusion or extract, and hydrogen peroxide, and optionally at least one stabilizing agent, such as glycerin. In some embodiments, an at least one plant infusion or extract is derived from a plant including woody-stemmed plants such as trees and shrubs, herbs, vegetables, fruits, agricultural crops, and ornamental plants and flowers. In particular embodiments, the plant includes components that possess mitigating and/or antioxidant properties.

in further embodiments, the components found in the plant include phenolic and polyphenol compounds (also phenolic compounds or polyphenols).

Phenolic compounds are characterized by having a main core formed by at least one phenol (hydroxybenzene) ring (such as phenolic acids) in which the hydrogen may be replaced by an active substituent such as hydroxyl, methyl or acetyl. Polyphenols refer to compounds that derive their basic structure from phenols and include multiple phenolic rings, and thus, are called polyphenols. Phenolic compounds can be natural or synthetic, that is, synthesized to mimic the structure of those that occur naturally. Accordingly, phenolic compounds used herein may be natural or synthetic compounds, or a combination of both. In any instance, phenolic and polyphenol compounds possess broad spectrum biological activity that includes antioxidant, anti-inflammatory, mitigating, antimicrobial, antimutagenic and/or anticarcinogenic activity.

Phenolic compounds are most commonly classified into two groups: flavonoid compounds and non-flavonoid compounds (also flavonoids and non-flavonoids). Flavonoids are the largest group of phenolic compounds found in plants. The main subclasses of flavonoids are the flavones, flavonols, flavan-3-ols, isoflavones, flavanones and anthocyanidins. The main subclasses of the other category of phenolics, i.e., the non-flavonoids, are phenolic acids, lignans, stilbenes, tannins, and lignins.

In particular embodiments, the plant including the phenolic and polyphenol compounds is selected from the group consisting of honeysuckle, sunflower, rose, lilac, lavender, wisteria, yarrow, chamomile, guava, peppermint, lemongrass, garlic, clove, ginger, geranium, cinnamon, rosemary, thyme, oregano, tarragon, green coffee bean (Coffea arabica), juniper, nutmeg, anise, noni, ginger, guava, pomegranate, artichoke, sage, jambolan, Cremaspora, St. John's Wort (Hypericum), parsley (Heteromorpha), cheesewoods (Pittosporum), Bolusanthus, Calpurnia, Maesa, Elaeodendron, African mulberry and combinations thereof. In further embodiments, the plant may be a member of the genus Lonicera. In other embodiments, the plant is a member of the species Lonicera caprifolium (goat-leaf honeysuckle, perfoliate honeysuckle), Lonicera periclymenum (European honeysuckle or woodbine), Lonicera japonica (Japanese honeysuckle, white honeysuckle, or Chinese honeysuckle), Lonicera sempervirens (coral honeysuckle or trumpet honeysuckle), Lonicera hildebrandiana (Burmese honeysuckle), Lonicera tartarica (Tartarian honeysuckle), Lonicera fragrantissima (winter honeysuckle), Lonicera pileate (privet honeysuckle), Lonicera nitida (box honeysuckle) and Lonicera syringantha (lilac-flowered honeysuckle).

In further embodiments, the at least one plant infusion or extract is derived from a honeysuckle plant, and in some embodiments, the at least one plant infusion or extract is derived from Lonicera japonica (Japanese honeysuckle). In particular embodiments, the at least one plant infusion or extract is derived from a bud, leaf, stem, blossom and/or fruit (Including the skin, flesh and seed of the fruit) of the plant. In particular embodiments, the portion of the plant from which the at least one plant infusion or extract is derived is organic. In some embodiments, the portion of the plant from which the at least one plant infusion or extract is derived is certified organic.

According to further embodiments of the compositions of the inventive concept, the at least one plant infusion or extract comprises a plant material derived from a flower bud, leaf, stem, blossom and/or fruit and a solvent. In some embodiments, the solvent is selected from the group consisting of water, alcohol, chloroform, acetone, petroleum ether, an ionic liquid and combinations thereof. In particular embodiments, the water is mineral water, spring water, tap water, deionized water, distilled water, sterile water, reverse osmosis water, water for injection (WFI) or a combination thereof. In some embodiments, the alcohol is methyl, ethyl, propyl, iso-propyl or butyl alcohol. As used herein, “ionic liquids” refer to salts with at least one organic cation and generally are restricted to salts with melting point below 100° C. Ionic liquids are frequently produced from ammonium, phosphonium or sulphonate ions.

In particular embodiments, the solvent is an organic solvent including being a certified organic solvent. In some embodiments, the organic solvent or certified organic solvent is water, ethanol, methanol, propanol, isopropanol, isopentane, n-pentane, n-hexane, dimethoxymethane, glycerol, benzyl alcohol, benzyloxyethanol, alkylene carbonates such as ethylene carbonate and propylene carbonate, phenoxyethanol, butanol, isobutanol, cyclohexane, cyclohexanol, ethylenecarbonate, ethyleneglycol monoethylether, ethylene glycol monobutyl ether, ethylene glycol monophenyl ether, 1-phenylethylalcohol, 2-phenylethylalcohol, o-methoxyphenol. In some embodiments of the composition, the at least one plant infusion or extract comprises 0.000-99.999% water, 0.000-99.999% alcohol and 0.001-99.999% plant material, and in some embodiments, 0.001-10% plant material. In particular embodiments, the alcohol is ethyl alcohol.

In some embodiments, the plant material is a flower bud or blossom. In particular embodiments, the plant material is honeysuckle flower buds.

In further embodiments, the at least one plant infusion or extract further comprises 0.001% to 99.999% phenolic compounds (phenolic acids and/or polyphenols). Particular phenolic compounds and/or polyphenols include the flavonoids such as the flavones, flavonols, flavan-3-ols, isoflavones, flavanones and anthocyanidins. In some embodiments, the phenolic compounds and/or polyphenol is a flavonoid. Exemplary flavonoids include, but are not limited to, quercetin, rutin, luteolin-7-O-β-D-glucopyranoside, kaempferol-3-O-β-D-glucopyranoside, apigenin-7-O-α-L-rhamnopyranoside, chrysoeriol-7-O-β-D-glucopyranosyl, luteolin-3′-L-rhamnoside, luteolin, flavoyadorinin-B, rhoifolin, quercetin-3-O-β-D-glucopyranoside, 3′-methoxy luteolin, 5,3′-dimethoxy luteolin, luteolin-5-O-β-D-glucopyranoside, apigenin, isorhamnetin-3-O-β-D-glucopyranoside, hyperoside, quercetin-7-O-β-D-glucopyranoside, kaempferol-3-O-β-D-rutinoside, isorhamnetin-3-O-β-D-rutinoside, 5-hydroxyl-3′,4′,7-trimethoxy flavone, 5-hydroxyl-6,7,8,4′-tetramethoxy flavone, corymbosin 5-hydroxyl-7,4′-dimethoxy flavone, lonicerin, 5,7,3′,4′,5′-pentamethoxy flavone, and 5,4′-dihydroxy-3′,5′-dimethoxy-7-β-D-glucoxy-flavone.

Other particular phenolic compounds and/or polyphenols include the non-flavonoids such as phenolic acids, lignans, stilbenes, tannins, and lignins. In some embodiments of the present inventive concept, the phenolic compound and/or polyphenol is a phenolic acid. Phenolic acids are generally phenols that include a phenolic ring and at least one carboxylic acid moiety. The main classifications of phenolic acids are hydroxycinnamic acids and hydroxybenzoic acids. According to embodiments of the present inventive concept, the phenolic compounds and/or polyphenols are hydroxycinnamic acids and/or hydroxybenzoic acids.

In particular embodiments, the phenolic compounds and/or polyphenols are hydroxycinnamic acids. The most commonly found naturally occurring hydroxycinnamic acid to date is chlorogenic acid. Chlorogenic acid (C₁₆H₁₈O₉; mol. wt.=354.31), is an ester of caffeic acid and quinic acid and is also referred to as cyclohexanecarboxylic acid, 3-[[3-(3,4-dihydroxyphenyl)-1-oxo-2-propenyl]oxy]-1,4,5-trihydroxy-, [1S-(1,3β,4,5)-(9CI), 3-Caffeoylquinic acid, 3-O-Caffeoylquinic acid, 3-(3,4-Dihydroxycinnamoyl)quinic acid, 3-O-(3,4-Dihydroxycinnamoyl)-D-quinic acid, [1S-(1,3β3,4,5)]-3-[[3-(3,4-Dihydroxyphenyl)-1-oxo-2-propenyl]oxy]-1,4,5-trihydroxycyclohexanecarboxylic acid NSC 407296, 1,3,4,5-Tetrahydroxycyclohexanecarboxylic acid 3-(3,4-dihydroxycinnamate) and trans-Chlorogenic acid. Other hydroxycinnamic acids include, but are not limited to, cinnamic acid, o-coumaric acid, m-coumaric acid, p-coumaric acid, ferulic acid, sinapic acid and caffeic acid. According to embodiments of the present invention, the phenolic compound and/or polyphenol is chlorogenic acid, cinnamic acid, o-coumaric acid, m-coumaric acid, p-coumaric acid, ferulic acid, sinapic acid, caffeic acid and derivatives thereof.

According to some embodiments, the phenolic compounds and/or polyphenols are hydroxybenzoic acids. Hydroxybenzoic acids are derived directly from benzoic acid. Exemplary hydroxybenzoic acids include, but are not limited to, benzoic acid, p-hydroxybenzoic acid, vanillic acid, gallic acid, protocatechuic acid, syringic acid, veratric acid and salicylic acid.

According to embodiments of the present inventive concept, the phenolic compound and/or polyphenol is benzoic acid, p-hydroxybenzoic acid, vanillic acid, gallic acid, protocatechuic acid, syringic acid, veratric acid, salicylic acid and derivatives thereof. According to further embodiments of the present inventive concept, the phenolic compound and/or polyphenol is a cinnamic acid derivative. In still further embodiments, the phenolic compound and/or polyphenol is chlorogenic acid and/or derivatives thereof.

As noted above, in addition to the compositions including at least one plant infusion or extract, the compositions include hydrogen peroxide and optionally at least one stabilizing agent, such as glycerin. In particular, hydrogen peroxide has been shown to provide an unexpected benefit of enhancing the preservation activity of the composition when compared to the activity of the composition without hydrogen peroxide. Moreover, the amount required to achieve this effect was minimal. Hydrogen peroxide can also deliver a desirable bleaching (or decolorizing) effect to the compositions. In particular embodiments, hydrogen peroxide is present in an amount ranging from about 0.001 to 10 wt %. In some embodiments, hydrogen peroxide is present in an amount ranging from about 0.001 to 5 wt %. In still some embodiments, hydrogen peroxide is present in an amount of about 2.0 wt %. In further embodiments, hydrogen peroxide is present in an amount of about 1.0 wt %. In still further embodiments, hydrogen peroxide is present in an amount of about 0.5 wt %.

At least one stabilizing agent may be present. In particular embodiments, the stabilizing agent possesses desirable solubilization characteristics, is stable under most conditions, can prevent crystallization at low temperatures and/or is nonvolatile. In some embodiments, solutes are more soluble in the stabilizing agent than water. For example, compounds such as tannins, alkaloids, phenols, thymols, and boric acids may be particularly soluble in the stabilizing agent when compared to water. In particular embodiments of the present inventive concept, the stabilizing agent can be selected from glycerin, propylene glycol, shea butter, jojoba, cocoa butter, avocado oil, coconut oil, olive oil, sunflower seed oil, grapeseed oil, almond oil, essential oils, etc. In particular embodiments, the stabilizing agent is glycerin. As used herein, “glycerin” (also called glycerol) is the chemical: 1,2,3-propanetriol and is a trihydric alcohol. In particular embodiments, glycerin is present in an amount ranging from about 0 to 90 wt %. In some embodiments, glycerin is present in an amount ranging from about 0 to 10 wt %. In other embodiments, glycerin is present in an amount of about 5 wt %. In still further embodiments, the stabilizing agent, including glycerin, is organic, and in some embodiments, certified organic.

In some embodiments of the present inventive concept, the at least one plant infusion or extract further comprises an organic acid, a flavonoid as described herein or a combination thereof. As used herein, an organic acid refers to an organic compound with acidic properties. The organic acid may be derived naturally, may be synthetic or a combination thereof. In particular embodiments, the organic acid is derived from a plant. In further embodiments, the organic acid is derived from a honeysuckle plant, and in some embodiments, the organic acid is derived from a Japanese honeysuckle. In still further embodiments, the organic acid is chlorogenic acid, isochlorogenic acid, caffeic acid, hexadecanoic acid, myristic acid, 3,5-O-dicaffeoylquinic acid, 4,5-O-dicaffeoylquinic acid, 3,4-O-dicaffeoylquinic acid, 1,3-O-dicaffeoylquinic acid, 3-ferulicoylquinic, 4-ferulicoylquinic, 5-O-caffeoylquinic acid, 4-O-caffeoylquinic acid, caffeoyl-CH₂—O-quinic acid, 1,5-O-dicaffeoylquinic acid, 1,4-O-dicaffeoylquinic acid, methylated dicaffeoylquinic acid, oleanolic acid, 3,5-O-dicaffeoylquinic acid methyl ester, methyl chlorogenate, 3-O-caffeoylquinic acid butyl ester, 3-O-caffeoylquinic acid, 3-caffeoylquinic acid methyl ester 3,5-dicaffeoylquinic acid butyl ester, vanillic acid, protocatechuic acid, chlorogenic acid butyl ester, chlorogenin tetraacetate, 5-feruloylquinic acids, methyl 3,5-di-O-caffeoylquinic acid, methyl 3,4-di-O-caffeoylquinic acid and caffeic acid methyl ester and isomers and derivatives thereof. In some embodiments, the organic acid is chlorogenenic acid, isochlorogenic acid, caffeic acid, hexadeanoic acid, myristic acid and isomers and derivatives thereof, or a combination thereof. In still other embodiments, the organic acid is chlorogenic acid or an isomer or derivative thereof.

Embodiments of the present invention further provide a composition comprising a phenolic compound and/or polyphenol, hydrogen peroxide and optionally at least one stabilizing agent, such as glycerin. Embodiments of the present invention also provide a composition comprising an organic acid and/or a flavonoid, hydrogen peroxide and optionally at least one stabilizing agent, such as glycerin. As noted above, the phenolic compound, polyphenol, organic acid and/or flavonoid may be natural, synthetic or a combination of both.

Embodiments of the present invention further provide plant infusions or extracts that comprise a solvent as described above, hydrogen peroxide and a plant material. In particular embodiments, the solvent is selected from the group consisting of water, alcohol, chloroform, acetone, petroleum ether, an ionic liquid and combinations thereof. In some embodiments, the plant material is derived from a member of the species Lonicera caprifolium (goat-leaf honeysuckle, perfoliate honeysuckle), Lonicera periclymenum (European honeysuckle or woodbine), Lonicera japonica (Japanese honeysuckle, white honeysuckle, or Chinese honeysuckle), Lonicera sempervirens (coral honeysuckle or trumpet honeysuckle), Lonicera hildebrandiana (Burmese honeysuckle), Lonicera tartarica (Tartarian honeysuckle), Lonicera fragrantissima (winter honeysuckle), Lonicera pileate (privet honeysuckle), Lonicera nitida (box honeysuckle) and Lonicera syringantha (lilac-flowered honeysuckle). In particular embodiments, the plant material is organic, and in particular, certified organic.

According to further embodiments of the invention, the plant infusion or extract is formed by adding a plant material to a solvent or combination of solvents; filtering the solution resulting from the addition of the plant material to the solvent or combination thereof, and adding hydrogen peroxide to the filtered solution, and optionally adding glycerin. In some embodiments, the plant material can be crushed, milled and/or dried before being contacted with the extraction solvent. The extraction can be assisted with stirring, shaking, agitating, circulating and/or heating. The extraction can be microwave, pressure and/or ultrasound assisted. In some embodiments, the solvent is selected from the group consisting of water, alcohol, chloroform, acetone, petroleum ether, an ionic liquid and combinations thereof. In further embodiments, the infusion or extract is formed by adding a plant material, such as a ground plant material, to a water and/or alcohol. In some embodiments, the alcohol is methyl, ethyl, propyl, iso-propyl or butyl alcohol. In particular embodiments, the starting material (i.e., the plant material, solvent and/or stabilizing agent(s)) is synthetic, natural, organic and/or a combination thereof. In some embodiments, the starting material is organic (preferably, certified organic) raw materials or at least 70%, 75%, 80%, 85%, 90%, 91%, 92%, 93%, 94%, 95%, 96%, 97%, 98% or 99% organic so that the final plant infusion or extract will be at least 70%, 75%, 80%, 85%, 90%, 91%, 92%, 93%, 94%, 95%, 96%, 97%, 98% or 99% organic. In particular embodiments, the final plant infusion or extract will be at least 90%, 91%, 92%, 93%, 94%, 95%, 96%, 97%, 98% or 99% organic. In particular embodiments, the final plant infusion or extract will be at least 95%, 96%, 97%, 98% or 99% certified organic.

Advantageously, the plant infusions or extracts can be certified as organic, using organic, and in particular, certified organic extraction solvents as described above and including, but not limited to, alcohols, water and ionic liquids. Accordingly, compositions and plant infusions or extracts described herein can be natural and/or organic. In particular embodiments, the compositions and plant infusions or extracts described herein are certified organic.

Further, the natural, organic and/or certified organic compositions and plant infusions or extracts described herein are substantially free of parabens, and in particular embodiments, paraben free. Parabens are man-made synthetic esters of p-hydroxybenzoic acid and have been used as a preservative in personal care, pharmaceutical and food products. Exemplary parabens include, but are not limited to, methylparaben, ethylparaben, propylparaben, butylparaben, heptylparaben, isobutylparaben, isopropylparaben, benzylparaben and their sodium salts. As used herein, “substantially free” generally refers to a level considered to be a trace amount or background level. Such levels do not cause material harm that may be associated with parabens. Further, substantially free also indicates that parabens have not been intentionally added to the compositions, plant infusions or extracts or methods described herein. In particular embodiments, substantially free refers to the presence of parabens at a concentration of about 0.010% or less or optionally about 0.001% or less. In particular embodiments of the present inventive concept, the natural, organic and/or certified organic compositions and plant infusions or extracts described herein do not contain any parabens.

Embodiments of the present invention further provide compositions and plant infusions or extracts that possess mitigating properties. In such instances, the compositions and plant infusions or extracts prevent or significantly minimize contamination by pathogenic and non-pathogenic microorganisms, suppress microbial growth, reduce microbial infestation, treat products or surfaces to improve product resistance to microbial infestation, reduce biofilm, prevent conversion of bacteria to biofilm, prevent or inhibit microbial infection, prevent spoilage, retard or minimize or prevent quorum sensing, and/or retard microbial reproduction. In particular embodiments, the mitigation properties of the compositions and infusions or extracts of the present invention allow the control of one or more specific microorganisms as described herein that are directly or indirectly infectious or pathogenic to humans (or both humans and animals).

In addition to mitigation properties, compositions and plant infusions or extracts of the present invention can also possess preservation properties. That is, the compositions and/or plant infusions or extracts can serve as a preservative to suppress microbial contamination by reducing, inhibiting or completely eliminating microbial growth and/or proliferation thereby protecting a product from, for example, degradation/putrefaction. The preservative compositions and plant extracts of the present invention impart a longer sustainable shelf-life, i.e., stability, reduction of contamination and/or infection and consistent product performance.

The preservative compositions and plant infusions or extracts may be used to reduce, inhibit or completely eliminate pathogen population in a variety of consumer products, such as personal care products, household, institutional and industrial (HI&I) care products, food products; beverages; therapeutics; and others. The compositions and plant infusions or extracts described herein may be used to partially or completely replace currently available chemicals, including synthetic chemicals, which are used as preservatives, some of which are known as toxic to humans and animals, or at least reduce their concentration in such products for human or animal use. In particular embodiments, the preservative compositions and plant infusions or extracts reduce the pathogen population and/or inhibit further microbial growth. According to some embodiments, the compositions and plant infusions or extracts described herein are natural and/or organic preservatives. In particular embodiments, the compositions and plant infusions or extracts described herein are certified organic preservatives.

The pathogens against which the compositions and plant infusions or extracts may be effective include a wide spectrum of microorganisms such as bacteria, viruses, virinos, viroids, phages, fungi (including yeasts and molds) lichens, spores, protozoa and some algae, and further include combinations of the same type of microorganism as well as combinations of different types of microorganisms.

In some embodiments, the microorganism is a bacterium. In other embodiments, the bacterium may be a gram-positive bacterium. In still other embodiments, the bacterium may be a gram-negative bacterium. In particular embodiments, the bacterium is selected from Bordetella pertussis, Borrelia burgdorferi, Brucella abortus, Brucella canis, Brucella melitensis, Brucella suis, Campylobacter jejuni, Chlamydia pneumonia, Chlamydia psittaci, Chlamydia trachomatis, Clostridium botulinum, Clostridium difficile, Clostridium perfringens, Clostridium tetani, Corynebacterium diphtheria, Enterococcus faecalis, Enterococcus faecium, Escherichia coli (E. coli), Enterotoxigenic Escherichia coli (ETEC), Enteropathogenic E. coli, Francisella tularensis, Haemophilus influenza, Helicobacter pylori, Legionella pneumophila, Leptospira interrogans, Listeria monocytogenes, Mycobacterium leprae, Mycobacterium tuberculosis, Mycoplasma pneumonia, Neisseria gonorrhoeae, Neisseria meningitidis, Pseudomonas aeruginosa, Rickettsia rickettsii, Salmonella typhi, Salmonella typhimurium, Shigella sonnei, Staphylococcus aureus, methicillin resistant Staphylococcus aureus (MRSA), Staphylococcus epidermidis, Staphylococcus saprophyticus, Streptococcus agalactiae, Streptococcus mutans, Streptococcus pneumonia, Streptococcus pyogenes, Treponema pallidum, Vibrio cholera, Vibrio harveyi, Yersinia pestis and combinations thereof.

In some embodiments, the microorganism is a virus. In particular embodiments, the virus is selected from Ebola and Marburg virus (Filoviridae); Semliki Forest virus, Ross River virus, chikungunya virus, O'nyong-nyong virus, Sindbis virus, eastern/western/Venezuelan equine encephalitis virus (Togaviridae, Alphavirus); rubella (German measles) virus (Togaviridae, Rubivirus); rabies virus, Lagos bat virus, Mokola virus (Rhabdoviridae, Lyssavirus); Amapari virus, Pichinde virus, Tacaribe virus, Junin virus, Machupo virus, Guanarito virus, Sabia virus, Lassa virus (Arenaviridae, Mammarenavirus); West Nile virus, dengue virus, yellow fever virus, Zika virus, Japanese encephalitis virus, St. Louis encephalitis virus, tick-borne encephalitis virus, Omsk hemorrhagic fever virus, Kyasanur Forest virus (Flaviviridae, Flavivirus); human hepatitis C virus (Flaviviridae, Hepacivirus); human immunodeficiency virus type 1 (Retroviridae, Lentivirus); influenza A/B/C virus (Orthomyxoviridae, the common “flu” virus); a corona virus (Coronaviridae, SARS-CoV, HCoV NL63, HCoV HKU1, MERS-CoV and SARS-CoV-2); respiratory syncytial virus (Paramyxoviridae, Pneumovirinae, Pneumovirus); Hendra virus, Nipah virus (Paramyxoviridae, Paramyxovirinae, Henipavirus); measles virus (Paramyxoviridae, Paramyxovirinae, Morbillivirus); variola major (smallpox) virus (Poxviridae, Chordopoxvirinae, Orthopoxvirus); human hepatitis B virus (Hepadnaviridae, Orthohepadnavirus); hepatitis delta virus (hepatitis D virus) (unassigned Family, Deltavirus); a papovavirus (e.g., papillomaviruses, including human papilloma virus (HPV)), a herpesvirus (e.g., herpes simplex virus, varicella-zoster virus, bovine herpesvirus-1, cytomegalovirus), a poxvirus (e.g., smallpox virus), a reovirus (e.g., rotavirus), a norovirus, a parvovirus (e.g., parvovirus B19, canine parvovirus), or a picornavirus (e.g., poliovirus, hepatitis A), or combinations thereof.

In some embodiments, the microorganism is a fungus, selected in some embodiments from Absidia corymbifera, Ajellomyces capsulatus, Ajellomyces dermatitidis, Arthroderma benhamiae, Arthrodermafulvum, Arthroderma gypseum, Arthroderma incurvatum, Arthroderma otae, Arthroderma vanbreuseghemii, Aspergillus flavus, Aspergillus fumigates, Aspergillus niger, Blastomyces dermatitidis, Candida albicans, Candida albicans var. stellatoidea, Candida dublinensis, Candida glabrata, Candida guilliermondii, Candida krusei, Candida parapsilosis, Candida pelliculosa, Candida tropicalis, Cladophialophora carrionii, Coccidioides immitis, Cryptococcus neoformans, Cunninghamella sp., Epidermophyton floccosum, Exophiala dermatitidis, Filobasidiella neoformans, Fonsecaea pedrosoi, Geotrichum candidum, Histoplasma capsulatum, Hortaea werneckii, Issatschenkia orientalis, Madurella grisae, Malasseziafurfur, Malasseziafurfur complex, Malassezia globosa, Malassezia obtuse, Malassezia pachydermatis, Malassezia restricta, Malassezia sloofflae, Malassezia sympodialis, Microsporum canis, Microsporum fulvum, Microsporum gypseum, Microsporum gypseum complex, Microsporum gypseum, Mucor circinelloides, Nectria haematococca, Paecilomyces variotii, Paracoccidioides brasiliensis, Penicillium marnefei, Phialophora verrucosa, Pichia anomala, Pichia guilliermondii, Pneumocystis jirovecii, Pseudallescheria boydii, Rhizopus oryzae, Rodotorula rubra, Saccharomyces cerevisiae, Scedosporium apiospermum, Schizophyllum commune, Sporothrix schenckii, Stachybotrys chartarum, Trichophyton mentagrophytes, Trichophyton mentagrophytes complex, Trichophyton mentagrophytes, Trichophyton mentagrophytes, Trichophyton rubrum, Trichophyton tonsurans, Trichophyton verrucosum, Trichophyton violaceum, Trichosporon asahii, Trichosporon cutaneum, Trichosporon cutaneum complex, Trichosporon inkin and Trichosporon mucoides.

In some embodiments, the fungus is yeast, being selected, in some embodiments, from Candida albicans, Candida albicans var. stellatoidea, Candida dublinensis, Candida glabrata, Candida guilliermondii, Candida krusei, Candida parapsilosis, Candida pelliculosa, Candida tropicalis, Cryptococcus neoformans, Filobasidiella neoformans, Geotrichum candidum, Issatschenkia orientalis, Malassezia furfur, Malassezia pachydermatis, Pichia anomala, Pichia guilliermondii, Pneumocystis jirovecii, Rodotorula rubra, Trichosporon asahii, Trichosporon cutaneum, Trichosporon inkin and Trichosporon mucoides.

In some embodiments, the fungus is mold, being selected, in some embodiments, from Absidia corymbifera, Arthroderma benhamiae, Arthrodermafulvum, Arthroderma gypseum, Arthroderma incurvatum, Arthroderma otae, Arthroderma vanbreuseghemii, Aspergillus favus, Aspergillusfumigates, Aspergillus niger, Cladophialophora carrionii, Coccidioides immitis, Epidermophytonfloccosum, Exophiala dermatitidis, Fonsecaea pedrosoi, Hortaea wereckii, Madurella grisae, Microsporum canis, Microsporum fulvum, Microsporum gypseum, Microsporum gypseum, Microsporum gypseum, Mucor circinelloides, Nectria haematococca, Paecilomyces variotii, Paracoccidioides brasiliensis, Penicillium marneffei, Pseudallescheria boydii, Rhizopus oryzae, Scedosporium apiospermum, Schizophyllum commune, Sporothrix schenckii, Stachybotrys chartarum, Trichophyton mentagrophytes complex, Trichophyton mentagrophytes, Trichophyton mentagrophytes, Trichophyton rubrum, Trichophyton tonsurans, Trichophyton verrucosum and Trichophyton violaceum.

In particular embodiments, the microorganisms are Escherichia coli, Staphylococcus aureus (including methicillin resistant Staphylococcus aureus), Pseudomonas aeruginosa Aspergillus brasiliensis and/or Candida albicams.

In particular embodiments, the microorganisms are non-pathogenic microorganisms. That is, the non-pathogenic microorganism does not typically cause disease and/or possesses attenuated pathogenicity.

The mitigating and/or preservation properties of the compositions and infusions or extracts of the present invention render the compositions and infusions or extracts as suitable cleaners or cleaning additives. “Clean” or cleaning,” as used herein, refers to the ability to perform or aid in soil (e.g., oil, pigment, food, blood, protein, bleachable, etc.) removal, dirt removal, dust removal, stain removal, odor removal, microbial population reduction, or a combination thereof.

Further, compositions and infusions or extracts of the present invention can serve as the active ingredient in a sanitizer. As used herein, “sanitizer” refers to a substance or composition that reduces, but not necessarily eliminates, microorganisms from the inanimate environment, including airborne microorganisms, to levels considered safe as determined by public health codes or regulations. Sanitizers include food contact and nonfood contact products as described herein.

Compositions and infusions or extracts of the present invention can also serve as an active ingredient in a disinfectant. As used herein, “disinfectant” refers to a substance or composition that destroys, neutralizes or inhibits the growth of disease-carrying microorganisms. Further, a disinfectant may be used in the air, on hard, inanimate surfaces and/or objects to destroy or irreversibly inactivate fungi and bacteria but not necessarily their spores. In particular embodiments, the disinfectant exhibits general or broad-spectrum disinfectant efficacy. Accordingly, in some embodiments, the subject matter of the present invention is a broad-spectrum disinfectant having efficacy against both gram-negative and gram-positive bacteria. In some embodiments, the compositions and infusions or extracts of the present invention may achieve total kill, at a log 6 reduction of organisms, at double the contact time required for sanitizing (i.e., 10 minutes versus 5 minutes). In further embodiments, the disinfectant compositions and infusions or extracts of the present invention may include virucidal properties and/or bactericidal properties. In some embodiments, the disinfectant product may be used on hard, non-porous surfaces, in the air and in the form of products as further described below. In still other embodiments, the disinfectant may be a high-level disinfectant, an intermediate-level disinfectant or a low-level disinfectant. “High-level disinfection” or “high-level disinfectant” refers to a compound or composition that kills substantially all organisms, except high levels of bacterial spores, and is effected with a chemical germicide cleared for marketing as a sterilant by the U.S. Food and Drug Administration. As used herein, the term “intermediate-level disinfection” or “intermediate-level disinfectant” refers to a compound or composition that kills mycobacteria, most viruses, and bacteria with a chemical germicide registered as a tuberculocide by the Environmental Protection Agency (EPA). As used herein, the term “low-level disinfection” or “low-level disinfectant” refers to a compound or composition that kills some viruses and bacteria with a chemical germicide registered as a hospital disinfectant by the EPA.

Embodiments of the present invention further provide products that include the compositions and/or plant infusions or extracts described herein or to which the compositions and/or plant infusions or extracts may be applied. In some embodiments, the products include a food, beverage, pharmaceutical, nutraceutical or other consumable products (e.g., cereal products, rice products, tapioca products, bread products, confectionery products, dessert products, gums, chewing gums, chocolates, ices, honey products, yeast products, baking-powder, salt and spice products, savory products, mustard products, vinegar products, sauces (condiments), tobacco products, cigars, cigarettes, processed foods, cooked fruit and vegetable products, meat and meat products, jellies, jams, fruit sauces, egg products, milk and dairy products, cheese products, butter and butter substitute products, milk substitute products, soy products, edible oils and fat products, alcoholic drinks, beers, soft drinks, mineral and aerated waters and other non-alcoholic drinks, fruit drinks, fruit juices, coffee, artificial coffee, tea, cocoa, sweeteners, pharmaceutical and non-pharmaceutical gums, tablets, lozenges, drops, emulsions, elixirs, syrups and other preparations for making beverages); cosmetic, agricultural treatment; water treatment; cleaning composition or consumer product. Exemplary non-limiting products include personal care products in the form of a gel, cream, ointment, lotion, solution and suspension; colon cleansing therapies, water-based paints, cutting oils; latex solutions; frozen foods; candy; canned products; pet foods; baby foods; water; beer; wine; ethanol-containing beverages; flower preservative; fruit preservative; fruit and/or vegetable wash; and sanitizer, disinfectant or bacteriocide agent applied in the air of an environment and/or onto a surface to be cleaned, sanitized and/or disinfected, including inanimate surfaces as well as human or animal skin, by various means including by washing, spraying, misting, fogging, wiping, etc., for example, a multi-surface cleaner, a fabric sanitizer, rinse-free hand soap, hand sanitizer or wipe such as a sprayed, coated or impregnated wipe.

In some embodiments, the compositions and/or plant infusions or extracts described herein can be used in a water treatment process. For example, the compositions and/or plant infusions or extracts described herein may be used to make a desired volume of water more suitable for a specific purpose. The compositions and/or plant infusions or extracts described herein can be added to any type of water in need of treatment to reduce, eliminate or kill various microorganisms that may be present in the water. The water may be drinking water, an industrial water supply, irrigation, recreational water, water that is returned to the environment, etc. The water treatment may be residential, commercial or industrial water treatment or purification. In particular embodiments, the water treatment is a portable water purification product and/or method where the compositions and/or plant infusions or extracts described herein can be carried and/or added to a water supply such as a personal water bottle or container as well as contaminated water in a disaster area and/or developing country.

As discussed above, in some embodiments, the product is a cleaning composition. In particular, the cleaning composition is a product for human or animal care and/or use. In further embodiments, the cleaning composition is a laundry detergent (liquid, solid, gel, paste, powder or any of these impregnated or otherwise incorporated onto a delivery substrate), laundry additive, fabric sanitizer, automatic or manual dishwashing detergent, multi-surface cleaner, rinse-free antibacterial hard surface soap, hard surface cleaner, hand soap, rinse-free hand soap, hand sanitizer, body wash, deodorizing spray, shampoo or animal care cleaning products such as shampoo, cage cleaner, cleaning wipes, rinse-free antibacterial hard surface soap, deodorizing spray, etc. In particular embodiments, the cleaning compositions are sulfate free, natural, organic and/or certified organic cleaning compositions including laundry detergents, stain removers, laundry pre-wash formulations, fabric sanitizers automatic or manual dishwashing detergents, multi-surface cleaners, hard surface cleaners, rinse-free antibacterial hard surface soaps, hand soaps, rinse-free hand soap, hand sanitizers, body wash, shampoo or animal cleaning products such as shampoo, cage cleaner, cleaning wipes, deodorizing spray, etc. See U.S. Pat. Nos. 9,447,366; 9,217,127; 9,217,125 and 8,835,370. That is, the presently disclosed cleaning compositions may be formulated to comply with the “organic” and “made with organic” guidelines of the United States Department of Agriculture's National Organic Program (NOP), while exhibiting commercially acceptable detergent properties. As used herein, the term “commercially acceptable detergent properties” refers generally to the degree of cleanliness, extent of effort, or both that a typical consumer would expect to achieve or expend when using a detergent product to address a typical soiling condition on a typical substrate, e.g., dishes or laundry, including clothing. This degree of cleanliness may, depending on the particular product and particular substrate, correspond to a general absence of visible soiling, or to some lesser degree of cleanliness. Cleanliness may normally be determined using generally agreed industry standard tests or localized variations of such tests. In the absence of such agreed industry standard tests, cleanliness may be evaluated using the test or tests already employed by a manufacturer or seller to evaluate the cleaning performance of its cleaning products sold in association with its brand.

Embodiments of the present invention further provide natural approaches to increasing stability, i.e., the shelf life of foods, beverages, household cleaners, cosmetics and other products as described above. That is, in addition to microbial susceptibility, such products may also be subject to oxidative degradation while on the shelf. The compositions and/or plant infusions or extracts described herein may provide a source of antioxidants, at least attributed to the presence of compounds such as flavonoids and polyphenols or phenolic acids (e.g. chlorogenic acid, caffeic acid, 4,5-dicaffeoylquinic acid, and 3,5-dicaffeoylquinic acid) as described herein in addition to embodiments that include glycerin (a non-phenolic), which may provide antioxidant properties.

Such antioxidant properties include, but are not limited to, imparting an inhibitory effect on the antioxidative reaction of product constituents, preventing oxidation, inhibiting oxygen free radical chain reactions and scavenging hydrogen peroxide, hydroxyl radical, and/or superoxide radicals. According to embodiments of the present invention, shelf life of a product may be extended. The extended shelf life is in particular upon storage at ambient temperature, substantially retains its initial flavor profile, color, texture characteristics, reduced bacterial deterioration, and/or is free from visible mold growth. In some embodiments, the shelf life of a product may be extended up to 1, 2 or 3 years by including compositions and/or plant infusions or extracts of the present invention therein. In particular embodiments, the shelf life of a product may be extended up to two (2) years. In certain embodiments, the shelf life of a product may be extended by one (1) year. In specific embodiments, the shelf life of a product may be extended by two (2) years.

In particular embodiments, in addition to being natural, organic and/or certified organic, the compositions and/or plant infusions or extracts described herein minimally alter the color, fragrance, and/or overall stability of any product unless to improve one or more of these properties.

The general procedures for making and using plant infusions or extracts and compositions and/or products including the same of the present invention can be readily understood and appreciated by one skilled in the art. Some aspects of the present invention are described in more detail in the following non-limiting examples. These examples are not intended to restrict the present invention, and may be modified within the range not deviating from the scope of this invention.

### EXAMPLES

The following examples have been included to provide guidance to one of ordinary skill in the art for practicing representative embodiments of the presently disclosed subject matter. In light of the present disclosure and the general level of skill in the art, those of skill can appreciate that the following examples are intended to be exemplary only and that numerous changes, modifications, and alterations can be employed without departing from the scope of the presently disclosed subject matter. The descriptions and specific examples that follow are only intended for the purposes of illustration, and are not to be construed as limiting in any manner to make compounds of the disclosure by other methods.

### Example 1a

### Preparation of Plant Extracts as Preservatives and/or Mitigating Agents

### Lab Scale Extraction and Preservative Test

Several plant extracts were prepared and tested for preservative efficacy, including ginger root, green coffee beans, rosemary leaves, and honeysuckle buds. The extracts were then dosed into an organic laundry formulation and tested for preservative efficacy against three unknown representative bacterial strains obtained from various batching facilities. In each case, the final extract was added to the organic laundry detergent as the last ingredient. Next, the dosed laundry detergent was split out into separate aliquots. Finally, each aliquot was inoculated with bacteria isolated from various batching facilities that represent different classifications including gram-positive cocci, gram positive bacilli, and gram negative rods (as determined from gram stains). The inoculated material was plated on tryptic soy agar at least two times between day 2 and day 28, with day 0 representing the inoculation time. In the representative examples listed in Table 1, dosages of 3-5% of each plant extract exhibited preservative efficacy.

The ginger extraction was prepared by mixing 150 g of 95% ethyl alcohol with 5 g ginger root powder and stirring for 30 minutes. This mixture was irradiated in a standard kitchen microwave at power level 3 for 2 minutes before cooling and filtering through a 0.45-micron filter. Finally, the filtrate was stored at 5° C.

The honeysuckle extraction was prepared by mixing 50 g of 95% ethyl alcohol with 5 g of ground dried honeysuckle buds and stirring on a hotplate set to 37° C. for 30 minutes. The mixture was then filtered through a 0.45 micron filter and the filtrate was set aside. Another 50 g of 95% ethyl alcohol was added to the same powder and re-extraction was run by putting the mixture at 37° C. overnight (approximately 18 hours). The mixture was filtered through a 0.45-micron filter and this filtrate was combined with the filtrate from the first extraction. The final filtrate was stored at 5° C.

The coffee bean extraction was prepared by mixing 173 g of distilled water with 30 g of ground green coffee beans and boiling while stirring for 10 minutes. The mixture was cooled to room temperature before filtering through a 0.45-micron filter and the filtrate was saved. The extraction was repeated by adding another 173 g distilled water to the ground coffee beans from the first step and stirring while boiling for 30 minutes. The mixture was filtered through a 0.45-micron filter after letting it cool to room temperature. All of the filtrates were combined together and stored at 5° C.

The rosemary extraction was prepared by mixing 50 g of preheated (45° C. oven for 1 hour) 95% ethyl alcohol with 5 g ground dried rosemary leaves. The mixture was stirred on a heated hotplate (set to 50° C.) for 30 minutes and then filtered through a 0.45-micron filter. The extraction was repeated 2 more times by adding another 50 g of preheated ethyl alcohol to the used rosemary leaves, stirring on a hotplate, and filtering again. All of the filtrates from the three extraction steps were combined in the end and stored at 5° C.

TABLE 1
Preservation Data in Organic Laundry Formulation for Several Plant Extracts

| | | | Unidentified | | | | | |
|---|---|---|---|---|---|---|---|---|
| | | Plant | Gram (+) | | Unidentified Gram | | Unidentified Gram | |
| | | Extract | Coccus | | (+) Bacillus | | (−) Rod | |
| | | Dosage in | Inoculum | Day 28 | Inoculum | Day 28 | Inoculum | Day 28 |
| Extract | Batch | Detergent | Level | Level | Level | Level | Level | Level |
| Type | Date | (Weight %) | (CFU/g) | (CFU/g) | (CFU/g) | (CFU/g) | (CFU/g) | (CFU/g) |
| Ginger | August 2017 | 5.000 | 2.45 × 10⁵ | <10 | 2.80 × 10⁵ | <10 | 4.38 × 10⁵ | <10 |
| Honey- | September 2017 | 5.000 | 2.45 × 10⁵ | <10 | 2.80 × 10⁵ | <10 | 4.38 × 10⁵ | <10 |
| suckle | | | | | | | | |
| Green | September 2017 | 5.0000 | 2.45 × 10⁵ | <10 | 2.80 × 10⁵ | <10 | 4.38 × 10⁵ | <10 |
| Coffee | | | | | | | | |
| Rosemary | October 2017 | 3.000 | 2.45 × 10⁵ | <10 | 2.80 × 10⁵ | <10 | 4.38 × 10⁵ | <10 |

### Example 1b

### Preparation of Organic Honeysuckle Extract and Extract Composition

### Lab Scale Extraction

The extraction solvent was prepared by mixing 142.86 g of 95% ethyl alcohol with 857.14 g distilled water in a 2-liter glass beaker and stirring until blended. This mixture was heated to 45° C. using either a heated magnetic stir plate or a heating chamber such as a lab oven, until a temperature of 45° C. was achieved.

20 g of ground organic honeysuckle buds (grind size of 40-60 mesh) were added to the preheated extraction solvent and stirred for 2 hours while maintaining a solution temperature of 45° C. A first filtration of the hot crude organic extract (filtrate) was made through a fine mesh sieve ( 1/32-inch mesh) and the honeysuckle solids were discarded. The organic extract underwent subsequent stepwise filtrations, beginning with a 100-mesh sieve, moving to a 200-mesh sieve, and finishing with a 325-mesh sieve. A final fine filtration step was performed using Whatman grade 113 paper (or 30-μm equivalent), followed by Whatman #4 paper (or 20-25-μm equivalent), and the final weight was recorded using an analytical balance.

The filtrate was further finished by placing it into a clean glass vessel, adding 2% by weight of a 50% hydrogen peroxide solution (based on final filtrate weight), and stirring for at least 30 minutes at 25° C. Lastly, 5% by weight of organic glycerin was added to the filtrate, and allowed to stir for at least 30 minutes, resulting in the final extract composition. The final extract composition was stored in a sealed, sterile glass, HDPE, or PETE bottle, and stored at 5° C. when not in use.

### Production Scale Extraction

The extraction solvent was prepared by charging 143.3 kg distilled water into a sanitized 175-gallon stainless steel tank equipped with a steam heating jacket, adding 23.6 kg of 95% ethyl alcohol, and mixing until fully dissolved. The mixture was heated to 45° C., with the temperature maintained for the duration of the extraction.

3.26 kg of ground organic honeysuckle flower buds (40-60 mesh) was weighed into a 10-micron sock filter, which was inserted into a stainless-steel filter housing. Using a 2-inch pneumatic diaphragm pump set to 100 psi, the solvent mixture was circulated through the filter housing and recirculated for a period of up to six (6) hours. The pump rate varied based on the back pressure exerted as the grounds became saturated and compacted in the filter and did slow throughout the extraction time. The circulation at 45° C. was maintained for up to six (6) hours, after which the filter bag was removed from the housing and the used grounds were discarded. A final filtration step was performed by inserting an empty half (0.5) micron filter bag into the filter housing, and starting the recirculation pump.

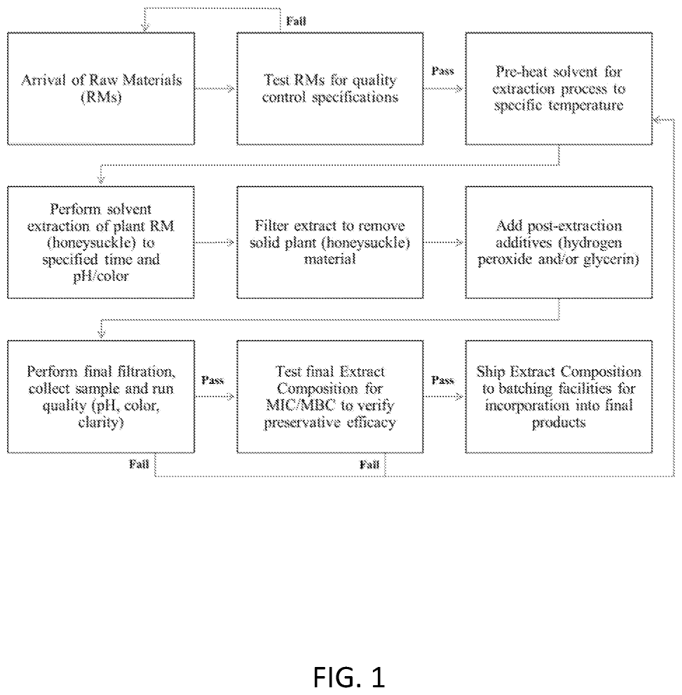

The filtrate was then finished in the tank by adding 3.08 kg of a 50% hydrogen peroxide solution, followed by 8.16 kg organic glycerin, and circulating the mixture for one (1) hour under the same pump settings as for the extraction. The final extract composition was then gravity fed through a half (0.5) micron filter bag and collected in a clean tote. The composition was stored at 5° C. when not in use. The overall process and a summary of the components that make up the composition are illustrated in FIG. 1 and listed in Table 2.

TABLE 2
Extract or Infusion Composition Summary

| | | Example | Example |
|---|---|---|---|
| | Example Range | Range by | Range by |
| Ingredient | by Weight (%) | Weight (%) | Weight (%) |
| Water | 0.000-99.999 | 65.000-95.000 | 70.000-85.000 |
| Organic Ethyl Alcohol | 0.000-99.999 | 2.000-20.000 | 5.000-15.000 |
| (95%) | | | |
| Organic Ground | 0.001-25.000 | 0.001-5.000 | .005-3.000 |
| Honeysuckle Flower | | | |
| Buds (~40-60 Mesh) | | | |
| Hydrogen Peroxide | 0.005-10.000 | 0.005-5.000 | 1.000-3.000 |
| (50%) | | | |
| Organic Glycerin | 0.000-90.000 | 0.000-10.000 | 1.00-7.000 |
| Total | 100 | 100 | 100 |

### Determining Content of Chlorogenic Acid and Derivatives

Identification of the primary chlorogenic acid derivatives in the extract composition (3,5-dicaffeoylquinic acid and 5 O-caffeoylquinic acid) was found using procedures outlined in Rapid Commun. Mass Spectrom. 2009; 23:363-383 and Brazilian J. Plant Physiol. Vol. 18 no. 1, 2006. The extract sample was weighed into an extraction vial and a methanol-water solvent was added to the extraction vial before sealing the vial. The extraction vial was placed in a water bath with sonication. After the allotted time of sonication, the sample was cooled and filtered.

The treated sample was injected into a high-performance liquid chromatography (HPLC) system equipped with a C18 column and a UV-Vis detector set at 330 nm (HPLC/UV-Vis/C18 column system was used with the chlorogenic acid standard solution in the range 5-250 ppm in methanol-water). Data were collected and analyzed using a methanol-water-acetic acid gradient using an external standard calibration method. The major chlorogenic acid peaks were identified based on the reference standard and the quantification of all the peaks were standardized to chlorogenic acid, with the results summarized in Table 3.

### Determining Content of Ethanol

The ethanol content in the extract composition was found by gas chromatography with flame-ionization detection (GC-FID, specifically a DB-624 60m 0.25 mm diameter 1.4 m film column, with standard solution 50-1000 ppm in DMSO). The sample was weighed into a volumetric flask and brought to volume with DMSO. The flask was mixed well, and an aliquot was transferred to a GC vial. For analysis, the sample was injected into a GC instrument equipped with a DB-624 column using helium as a carrier gas. The analysis was performed using an oven temperature gradient to achieve appropriate separation of the analytical peak(s). Data were collected and analyzed using a chromatography workstation based on an external standard calibration method. The ethanol peak was identified based on retention time of the standards, with the results summarized in Table 3.

### Determining Content of Hydrogen Peroxide

The extract composition was analyzed for hydrogen peroxide content using a Pierce Quantitative Peroxide Kit, aqueous-compatible formulation (Part #23280), available from ThermoFisher Scientific.

The test detects peroxide based on oxidation of ferrous to ferric ion in the presence of xylenol orange. In this assay, peroxide first reacts with sorbitol, converting it to a peroxyl radical, which in turn initiates Fe²⁺ oxidation to Fe³⁺. In a sulfuric acid solution, the Fe³⁺ complexes with the xylenol orange dye to yield a purple product with maximum UV absorbance at 560 nm. The molar extinction coefficient of the xylenol orange-Fe³⁺ complex is 1.5×10⁴ M⁻¹ cm⁻¹ in 25 mM H₂SO₄ at room temperature. The complex in both assays absorbs strongest between 540 and 580 nm but absorbs measurably up to 620 nm. When using microplates, the best wavelength for measurement is 595 nm (best signal-to-noise). The maximum absorbance of the assay components before addition of peroxide is at 430 nm.

Peroxide standards were prepared by serially diluting a 30% (8.8M) hydrogen peroxide stock solution to achieve 8 standards in the concentration range of 1-1000 μM. The high standard (1000 μM) was made by adding 100 μL of 30% hydrogen peroxide to 880 ml of ultrapure water or buffer.

1 ml of working reagent solution was prepared for each sample to be assayed in spectrophotometer cuvettes. For each working reagent, 1 volume of reagent A (25 mM ammonium ferrous (II) sulfate, 2.5M H₂SO₄) was mixed with 100 volumes of Reagent B (100 mM sorbitol, 125 μM xylenol orange in water).

For the spectrophotometer measurements, 10 volumes of working reagent (WR) were added to 1 volume of sample, and mixed. Samples were incubated for 20 minutes at room temperature to ensure reaction endpoints were reached.

Absorbance was measured at 560 nm (optimal wavelength in a spectrophotometer; 595 nm is optimal if using a plate reader). The concentration of peroxide in the sample was calculated by comparison to the standard curve after curve-fitting the standards. As expected, the assay returned results of approximately 1.0% for hydrogen peroxide content in the finished extract composition, as shown in Table 3.

TABLE 3
Composition Assay and Extraction Yield

| | | | 3,5- | 5 O- | | | |
|---|---|---|---|---|---|---|---|
| | | | dicaffeoyl- | caffeoyl- | | | |
| | | | quinic | quinic | 5-CQA + | | |
| | | | acid (3,5- | acid (5- | 3,5- | Ethyl | Hydrogen |
| Extract | Batch | Test | diCQA) | CQA) | diCQA | Alcohol | Peroxide |
| Type | Date | Date | (mg/g)* | (mg/g)* | (mg/g)* | (%) | (%) |
| Lab Extract- | Sep. 27, 2018 | Oct. 2, 2018 | 0.211 | 0.508 | 0.719 | 13.70 | 0.929 |
| Fresh | | | | | | | |
| Lab Extract- | Sep. 27, 2018 | Dec. 5, 2018 | 0.0248 | 0.193 | 0.218 | N/A | N/A |
| Aged at 25° C. | | | | | | | |
| Pilot Extract | Nov. 28, 2018 | Dec. 5, 2018 | 0.0385 | 0.0821 | 0.121 | N/A | N/A |
| 1-1-Hour | | | | | | | |
| Time Point | | | | | | | |
| Pilot Extract | Nov. 28, 2018 | Dec. 5, 2018 | 0.0487 | 0.107 | 0.156 | N/A | N/A |
| 1-3-Hour | | | | | | | |
| Time Point | | | | | | | |
| Pilot Extract | Nov. 28, 2018 | Dec. 5, 2018 | 0.0565 | 0.127 | 0.184 | 10.52 | N/A |
| 1-Final | | | | | | | |
| Retain | | | | | | | |
| Pilot Extract | Mar. 20, 2019 | Mar. 26, 2019 | 0.0537 | 0.175 | 0.229 | N/A | N/A |
| 2-1-Hour | | | | | | | |
| Time Point | | | | | | | |
| Pilot Extract | Mar. 20, 2019 | Mar. 26, 2019 | 0.0620 | 0.203 | 0.265 | N/A | N/A |
| 2-3-Hour | | | | | | | |
| Time Point | | | | | | | |
| Pilot Extract | Mar. 20, 2019 | Mar. 26, 2019 | 0.0410 | 0.199 | 0.240 | N/A | N/A |
| 2-Final 6- | | | | | | | |
| Hour | | | | | | | |
| Full Batch | Sep. 20, 2019 | Sep. 24, 2019 | 0.0463 | 0.0919 | 0.138 | 12.02 | N/A |
| Certified | | | | | | | |
| Organic | | | | | | | |
| Extract-3- | | | | | | | |
| Hour Time | | | | | | | |
| Point | | | | | | | |
| Full Batch | Sep. 20, 2019 | Sep. 24, 2019 | 0.0614 | 0.118 | 0.179 | 13.13 | N/A |
| Certified | | | | | | | |
| Organic | | | | | | | |
| Extract- | | | | | | | |
| Final 6-Hour | | | | | | | |

*Error: +/−1.6% to +/−10% of reported value, depending on specific assay

### Example 2

### Antimicrobial Characteristics: MIC and MBC and Preservation Efficacy

The Clinical and Laboratory Standards Institute (CLSI) has established protocols and standards for establishing MIC and MBC in products. A common methodology utilized for MIC is CISI M07-A9, Methods for Dilution Antimicrobial Susceptibility Tests for Bacteria That Grow Aerobically. CLSI also has developed methods specific for the yeasts, filamentous fungi, and anaerobic bacteria. For MBC determination, CLSIM26-A, Methods for Determining Bactericidal Activity of Antimicrobial Agents, is an accepted industry standard.

The Minimum Inhibitory Concentration (MIC) is defined as the lowest concentration of an antimicrobial ingredient or agent that is bacteriostatic (prevents the visible growth of bacteria). MICs are used to evaluate the antimicrobial efficacy of various compounds by measuring the effect of decreasing concentrations of antimicrobial agent over a defined period in terms of inhibition of microbial population growth.

Various concentrations of the compounds are inoculated with cultured bacteria, and the results are measured using agar dilution or broth dilution (macro or micro) to determine at what level the MIC endpoint is established. Susceptibility testing is typically conducted using organisms that contribute to an infectious process warranting antimicrobial chemotherapy. The samples were tested against five microorganisms required for Category 2. A commonly used cocktail of bacteria, mold and yeast for household and personal cam pathogens is:
Escherichia coli (ATCC8739)—Gram-negative, rod-shaped, facultative anaerobic coliform
Staphylococcus aureus (ACC 6538)—Gram-positive, spherical-shaped, facultative anaerobe
Pseudomonas aeruginosa (ATCC 9027)—Gram-negative, rod-shaped microorganism with a single flagellum
Aspergillus brasiliensis (ATCC 16404)—Conidiophore (a sexual spore generating aerobic fungus)
Candida albicans (ATCC 10231)—Facultative aerobic fungus that can grow both as a yeast and as a filamentous fungus

The Minimum Bactericidal Concentration (MBC) is the lowest concentration of an antimicrobial agent required to kill a microorganism over a fixed, somewhat extended period, such as 18 hours or 24 hours, under a specific set of conditions. It can be determined from the broth dilution of MIC tests by sub-culturing to agar plates that do not contain the test agent. The MBC is identified by determining the lowest concentration of antibacterial agent that reduces the viability of the initial bacterial inoculum by a pre-determined reduction such as ≥99.9%. The MBC is complementary to the MIC; whereas the MIC test demonstrates the lowest level of antimicrobial agent that greatly inhibits growth, the MBC demonstrates the lowest level of antimicrobial agent resulting in microbial death. If a MIC shows inhibition, plating the bacteria onto agar might still result in organism proliferation because the antimicrobial did not cause death. Antibacterial agents are usually regarded as bactericidal if the MBC is no more than four times the MIC.

Suspensions of test microorganisms were standardized by dilution in Mueller-Hinton broth. The extract composition was diluted to 4% (v/v) for bacterial testing and 16% (v/v) for fungal testing in sterile phosphate buffered saline (PBS) solution prior to use in the study. For the pH study, the PBS solution used for dilution was pH adjusted to a pH of 4, 6, 8 and 10 using sodium hydroxide or hydrochloric acid, as appropriate, prior to test substance dilution for testing. The test substances were prepared by conducting serial 1:1 dilutions in a 96-well microtiter plate, with the initial concentration at 2% (v/v) for bacterial testing and 8% (v/v) for fungal testing. All wells containing diluted test substances were inoculated with test microorganisms, individually. The microtiter plates were incubated for 24 hours. After the incubation period, observations were made to determine the minimum concentration of test substance in a well exhibiting no turbidity (with turbidity indicative of test microorganism growth). A summary of the MIC and MBC test parameters is listed in Table 4.

TABLE 4
MIC and MEC Study Test Parameters

| | |
|---|---|
| Test Substance Concentrations, Bacteria | 2000-0.0039% |
| Test Substance Concentrations, Fungi | 8.000-0.0156% |
| Final Well Volumes | 0200 ml |
| Replicates | Single |
| Culture Growth Media/Growth Time | Tryptic Soy Broth/24 |
| | hours or 7 days |
| Culture Dilution Media/Test | Mueller Hinton Broth/ |
| Dilution Media | Mueller Hinton Broth |
| Inoculum Concentration/Inoculum Volume | 1 × 10⁶ CRU/ml/0.100 ml |
| Incubation Temperature/Incubation Time | 36 +/− 1° C./18-24 hours |
| Enumeration Plate Temperature/Incubation | 36 +/− 1° C./ |
| Time | 24-48 hours |

The following criteria for test validity were met:
The average number of viable bacteria recovered at time zero from the growth control wells needed to be approximately 1×10⁵ cells/ml or greater.
Positive controls demonstrated growth of appropriate test microorganisms.
Negative controls demonstrated no growth of test microorganisms.
   The results in Tables 5a-c demonstrate that the extract composition provides effective bactericidal and fungal protection for aqueous-based compositions at relatively low usage levels (0.25-0.50 wt/), and across abroad pH range, for example, pH 4-10. For aggressive molds, the usage level is significantly higher, suggesting that supplemental mold protection would be needed for lower-pH, high moisture, or other formulations that would be susceptible to mold growth.

### Tables 5a-c: Antimicrobial Characteristics of Extract Composition, Varying Extract Batch and MIC/MBC Testing pH

TABLE 5a
Antimicrobial Characteristics of Extract Composition

| | | | | | Log₁₀ Reduction | | | |
|---|---|---|---|---|---|---|---|---|
| | | | | | Compared to | | | |
| | MIC Bacterial | | MBC Bacterial | | Control at T = 0 | | MIC Fungal | |
| | Strains (%) | | Strains (%) | | Using MBC | | Strains (%) | |
| | S. | P. | S. | P. | S. | P. | C. | A. |
| | aureus | aeruginosa | aureus | aeruginosa | aureus | aeruginosa | albicans | brasiliensis |
| Extract Type | 6538¹ | 9027² | 6538¹ | 9027² | 6538¹ | 9027² | 10231³ | 16404⁴ |
| Lab Extract-4 | 0.125 | 0.50 | 0.25 | 0.50 | >4.59 | >4.50 | 2 | 8 |
| Month Aged | | | | | | | | |
| (25° C.) | | | | | | | | |
| Pilot Extract #1- | 0.125 | 0.25 | 0.25 | 0.25 | >4.59 | >4.50 | 2 | 8 |
| 6-Hour Extinction- | | | | | | | | |
| 2 Month Aged | | | | | | | | |
| (5° C.) | | | | | | | | |
| Pilot Extract #1- | 0.125 | 0.25 | 0.25 | 0.25 | 4.59 | >4.50 | 2 | 8 |
| 6-Hour Extinction- | | | | | | | | |
| 2 Month Aged | | | | | | | | |
| (25° C.) | | | | | | | | |
| Pilot Extract #1- | 0.125 | 0.50 | 0.25 | 0.50 | 4.59 | >4.50 | 2 | 8 |
| 6-Hour Extinction- | | | | | | | | |
| 2 Month Aged | | | | | | | | |
| (45° C.) | | | | | | | | |

¹initial contamination level = 3.85 × 10⁵
²initial contamination level = 3.15 × 10⁵
³initial contamination level = 3.25 × 10⁵
⁴initial contamination level = 8.90 × 10⁵

TABLE 5b
pH Range of Extract Composition Antimicrobial Efficacy

| | MIC Bacterial | | MBC Bacterial | | MIC Fungal Strains | | MBC Fungal | |
|---|---|---|---|---|---|---|---|---|
| | Strains (%) for pH | | Strains (%) for | | (%) for pH range 4- | | Strains (%) for pH | |
| | range 4-10 | | pH range 4-10 | | 10 | | range 4-10 | |
| | S. | P. | S. | P. | C. | A. | C. | A. |
| | aureus | aeruginosa | aureus | aeruginosa | albicans | brasiliensis | albicans | brasiliensis |
| Extract Type | 6538¹ | 9027² | 6538¹ | 9027² | 10231³ | 16404⁴ | 10231³ | 16404⁴ |
| Pilot Extract #1- | 0.25 | 0.5 | 0.25- | 0.5 | 2 | >8% | 4 | >8% |
| 6-Hour Extraction- | | | 0.5 | | | | | |
| 4 Month Aged | | | | | | | | |
| (5° C.) | | | | | | | | |
| Pilot Extract #2- | 0.25 | 0.5 | 0.250 | 0.5 | 2 | >8% | 4 | >8% |
| 6-Hour Extraction | | | | | | | | |

¹initial contamination level = 3.85 × 10⁵
²initial contamination level = 3.15 × 10⁵
³initial contamination level = 3.25 × 10⁵
⁴initial contamination level = 8.90 × 10⁵

TABLE 5c
Complete Summary of Extract Composition Antibacterial Characteristics

| | | | | S. aureus | | P. aeruginosa/ | |
|---|---|---|---|---|---|---|---|
| | | Lot | | 6538 | | 9027 | |
| Extract Type* | Batch Date | Number | Test Date | MIC | MBC | MIC | MBC |
| Lab Extract Aged at | Aug. 23, 2018 | 10-73a | Nov. 18, 2019 | 0.250 | 0.500 | 0.250 | 0.250 |
| 25° C. (+5% EtOH) | | | | | | | |
| Lab Extract Aged at | Aug. 23, 2018 | 10-73b | Nov. 18, 2019 | 0.500 | 1.000 | 0.5 | 0.5 |
| 45° C. (+5% EtOH) | | | | | | | |
| Lab Extract Aged at | Sep. 11, 2018 | 10-78a | Sep. 26, 2019 | 0.063 | 0.125 | 0.250 | 0.250 |
| 25° C. (0 glycerin) | | | | | | | |
| Lab Extract Aged at | Sep. 11, 2018 | 10-78a | Sep. 26, 2019 | 0.125 | 0.125 | 0.250 | 0.500 |
| 25° C. (5% glycerin) | | | | | | | |
| Lab Extract Aged at | Sep. 27, 2018 | 10-83c | Jan. 21, 2019 | 0.125 | 0.250 | 0.250 | 0.250 |
| 5° C. | | | | | | | |
| Lab Extract Aged at | Sep. 27, 2018 | 10-83c | Jan. 21, 2019 | 0.125 | 0.250 | 0.500 | 0.500 |
| 25° C. | | | | | | | |
| Lab Extract Aged at | Sep. 27, 2018 | 10-83c | Jan. 21, 2019 | 0.125 | 0.250 | 0.250 | 0.500 |
| 45° C. | | | | | | | |
| First Pilot Extract 1- | Nov. 28, 2018 | 2018- | Jan. 21, 2019 | 0.125 | 0.250 | 0.250 | 0.250 |
| Hour Time Point | | 08107 | | | | | |
| First Pilot Extract 3- | Nov. 28, 2018 | 2018- | Jan. 21, 2019 | 0.125 | 0.250 | 0.250 | 0.250 |
| Hour Time Point | | 08107 | | | | | |
| First Pilot Extract | Nov. 28, 2018 | 2018- | Jan. 21, 2019 | 0.125 | 0.125- | 0.250 | 0.250- |
| Final 6-Hour | | 08107 | | | 0.250 | | 0.500 |
| First Pilot Extract | Nov. 28, 2018 | 2018- | Mar. 25, 2019 | 0.250 | 0.250 | 0.500 | 0.500 |
| Final 6-Hour Aged at | | 08107 | | | | | |
| 25° C. | | | | | | | |
| First Pilot Extract | Nov. 28, 2018 | 2018- | Sep. 26, 2019 | 0.125 | 0.125 | 0.250 | 0.500 |
| Final 6-Hour Aged at | | 08107 | | | | | |
| 25° C. | | | | | | | |
| First CO Extract Final | Mar. 20, 2019 | 2019- | Mar. 25, 2019 | 0.125 | 0.125- | 0.250 | 0.500 |
| 6-Hour | | 08770 | | | 0.250 | | |
| First CO Extract Final | Mar. 20, 2019 | 2019- | Jul. 29, 2019 | 0.125 | 0.250 | 0.250 | 0.500 |
| 6-Hour Aged at 5° C. | | 08770 | | | | | |
| First CO Extract Final | Mar. 20, 2019 | 2019- | Sep. 26, 2019 | 0.125 | 0.125 | 0.250 | 0.500 |
| 6-Hour Aged at 25° C. | | 08770 | | | | | |
| First CO Extract Final | Mar. 20, 2019 | 2019- | Feb. 5, 2020 | 0.250 | 0.250 | 0.500 | 0.500 |
| 6-Hour Aged at 25° C. | | 08770 | | | | | |
| First CO Extract Final | Mar. 20, 2019 | 2019- | Apr. 23, 2020 | 0.250 | 0.500 | 0.500 | 0.500 |
| 6-Hour Aged at 25° C. | | 08770 | | | | | |
| Lab Extract (+1% | Jul. 24, 2019 | 17-32a | Jul. 29, 2019 | 0.125 | 0.250 | 0.250 | 0.500 |
| Peroxide) | | | | | | | |
| Lab Extract (+0.5% | Jul. 24, 2019 | 17-32b | Jul. 29, 2019 | 0.250 | 0.250 | 0.500 | 0.500 |
| Peroxide) | | | | | | | |
| Second CO Extract 3- | Sep. 20, 2019 | 2019- | Sep. 26, 2019 | 0.125 | 0.125 | 0.250 | 0.250 |
| Hour Time Point | | 09769 | | | | | |
| Second CO Extract | Sep. 20, 2019 | 2019- | Sep. 26, 2019 | 0.125 | 0.125 | 0.250 | 0.500 |
| Final 6-Hour | | 09769 | | | | | |
| Second CO Extract | Sep. 20, 2019 | 2019- | Apr. 23, 2020 | 0.250 | 0.250 | 0.500 | 0.500 |
| Final 6-Hour Aged at | | 09769 | | | | | |
| 25° C. | | | | | | | |
| Lab Extract (+5% | Oct. 9, 2019 | 17-56a | Oct. 31, 2019 | 0.250 | 0.500 | 0.500 | 0.500 |
| EtOH, 0 Glycerin) | | | | | | | |
| Lab Extract (+5% | Oct. 9, 2019 | 17-56b | Oct. 31, 2019 | 0.250 | 0.500 | 0.500 | 0.500 |
| EtOH, 5% Glycerin) | | | | | | | |
| Lab Extract (+8% | Oct. 9, 2019 | 17-56c | Oct. 31, 2019 | 0.250 | 0.500 | 0.500 | 0.500 |
| EtOH, 0 Glycerin) | | | | | | | |
| Lab Extract (+8% | Oct. 9, 2019 | 17-56d | Oct. 31, 2019 | 0.250 | 0.500 | 0.500 | 0.500 |
| EtOH, 5% Glycerin) | | | | | | | |
| Third CO Extract 3- | Oct. 11, 2019 | 2019- | Oct. 31, 2019 | 0.250 | 0.500 | 0.500 | 0.500 |
| Hour Time Point | | 09898 | | | | | |
| Third CO Extract Final | Oct. 11, 2019 | 2019- | Oct. 31, 2019 | 0.250 | 0.250 | 0.500 | 0.500 |
| 6-Hour | | 09898 | | | | | |
| Third CO Extract Final | Oct. 11, 2019 | 2019- | Feb. 5, 2020 | 0.250 | 0.250 | 0.500 | 0.500 |
| 6-Hour Aged at 25° C. | | 09898 | | | | | |
| Fourth CO Extract | Nov. 7, 2019 | 2019- | Nov. 18, 2019 | 0.250 | 0.500 | 0.250 | 0.250 |
| Final 6-Hour | | 10067 | | | | | |
| Fifth CO Extract Final | Jan. 30, 2020 | 2020- | Feb. 5, 2020 | 0.250 | 0.250 | 0.500 | 0.500 |
| 6-Hour | | 10466 | | | | | |
| Sixth CO Extract Final | Feb. 3, 2020 | 2020- | Feb. 5, 2020 | 0.250 | 0.250 | 0.500 | 0.500 |
| 6-Hour | | 10527 | | | | | |
| Seventh CO Extract | Mar. 21, 2020 | 2020- | Apr. 23, 2020 | 0.250 | 0.250 | 0.250 | 0.250 |
| Final 6-Hour | | 10669 | | | | | |
| Eighth CO Extract | Mar. 25, 2020 | 2020- | Apr. 23, 2020 | 0.250 | 0.250 | 0.250 | 0.250 |
| Final 6-Hour | | 10673 | | | | | |

*CO = certified organic

### Preservation Challenge Data

In addition to assessing the overall antimicrobial characteristics of the Composition, the Composition was further evaluated in several finished natural cleaning formulations and found to effectively preserve a variety of HI&I formulations against the microorganisms listed, as exemplified in Examples 3-6 below.

The USP 51 Challenge Test is a 28-day test used to verify the effectiveness of a preservative system in a finished product formulation. The test organisms are pooled into two groups, a bacterial mixture (E. coli, S. aureus, P. aeruginosa) and a fungal mixture (C. albicans, A. brasiliensis).

Prior to initiating the Challenge Test, the sample was initially tested for aerobic bacteria, yeast, and mold following the USP <51> guidelines. This initial screen was imperative to ensure the product did not contain any microorganisms prior to beginning the inoculations. No organisms were found in the samples during the initial screen. Pure culture challenge yields specific data on each microorganism employed in the study. For each test microorganism, results are presented as contact time (days) and colony counts, expressed as colony forming units per mass or volume, or CFU/g or ml.

Where possible, the Challenge Test was also completed for the same formulation dosed with a typical level of a commercially available synthetic preservative, for comparison against the preservation characteristics of the Composition in the same formulation.

### Effectiveness Standards:

Bacteria: Not less than 2.0 log reduction from the initial count at 14 days, and no increase from the 14-day count at 28 days.
Yeast and Molds: No increase from the initial calculated count at 14 and 28 days.
Upon re-inoculation, the same effectiveness standards apply.

### Example 3a

### Natural All-Purpose Cleaner

Ingredient List—Water, Caprylyl/Decyl Glucoside, Honeysuckle Extract Composition, Citric acid
Finished formula pH is near neutral (range of 6.0-8.0)

| Trade Name | Weight % | INCI Name | Manufacturer |
|---|---|---|---|
| Glucopon 215 | 1.00-5.00 | Caprylyl/Decyl | BASF |
| UP | | Glucoside | Corporation, |
| | | | Florham |
| | | | Park, NJ |
| Water | 89.00-98.75 | Water | |
| Citric Acid, | 0-2.00 | Citric Acid | Jungbunzlauer, |
| Anhydrous | | | Inc, Newton |
| | | | Centre, MA |
| Honeysuckle | 0.25-4.00 | Water (and) Ethyl | Greenology |
| Extract | | Alcohol (and) | Products, LLC, |
| Composition | | Lonicera Japonica | Raleigh, NC |
| | | (Honeysuckle) | |
| | | Flower (and) | |
| | | Glycerin (and) | |
| | | Hydrogen | |
| Total | 100.00 | Peroxide | |

### Preservative Challenge Results

Natural All-Purpose Cleaner With < 4.00%
(Honeysuckle Extract) Composition

| | Inoculum | 0 | Day | Day | Day | Day |
|---|---|---|---|---|---|---|
| Test Organism | Level (CFU/g) | Hours | 7 | 14 | 21 | 28 |
| E. coli | 3.1 × 10⁶ | 2500 | <10 | <10 | <10 | <10 |
| P. aeruginosa | 3.0 × 10⁶ | 2100 | <10 | <10 | <10 | <10 |
| S. aureus | 3.4 × 10⁶ | 1700 | <10 | <10 | <10 | <10 |
| C. albicans | 3.3 × 10⁵ | 2000 | <10 | <10 | <10 | <10 |
| A. (niger) | 5.8 × 10⁵ | 6900 | <10 | <10 | <10 | <10 |
| brasiliensis | | | | | | |

This study clearly demonstrated that the effectiveness of the honeysuckle extract composition, in a generally neutral pH cleaning formulation, against E. coli, P. aeruginosa, P. aureus, A. brasiliensis and C. albicans meets the Category 2 requirement, which is no less than 2.0 log₁₀ reduction from the initial count at 14 days and no increase from the 14-day count at 28 days for bacteria, and no increase from the initial calculated count at 14 and 28 days for yeast and mold.

For efficacy comparison, the same formulation was preserved with and tested against a known synthetic preservative, benzisothiazolone, in place of the honeysuckle extract composition, at a level within the manufacturer's recommended dosage range.

Ingredient List—Water, Caprylyl/Decyl Glucoside, Benzisothiazolinone, Citric Acid Finished formula pH is near neutral (range of 6.0-8.0)

| Trade Name | Weight % | INCI Name | Manufacturer |
|---|---|---|---|
| Glucopon | 1.00-5.00 | Caprylyl/Decyl | BASF Corporation, |
| 215 UP | | Glucoside | Florham Park, NJ |
| Water | 92.00-98.90 | Water | |
| Citric Acid, | 0-2.00 | Citric Acid | Jungbunzlauer, Inc, |
| Anhydrous | | | Newton Centre, MA |
| Proxel AQ | 0.10-1.00 | Benziso- | Lonza, |
| (BIT) | | thiazolinone | Alpharetta, GA |
| Total | 100.00 | | |

### Preservative Challenge Results

Natural All-Purpose Cleaner With < 1.00% BIT

| | Inoculum | 0 | Day | Day | Day | Day |
|---|---|---|---|---|---|---|
| Test Organism | Level (CFU/g) | Hours | 7 | 14 | 21 | 28 |
| E. coli | 3.7 × 10⁶ | <10 | <10 | <10 | <10 | <10 |
| P. aeruginosa | 3.1 × 10⁶ | 490 | <10 | <10 | <10 | <10 |
| S. aureus | 2.6 × 10⁶ | <10 | <10 | <10 | <10 | <10 |
| C. albicans | 3.3 × 10⁵ | 790 | 10 | <10 | <10 | <10 |
| A. (niger) | 1.0 × 10⁶ | 670 | 320 | <10 | <10 | <10 |
| brasiliensis | | | | | | |

### Example 3b

### Natural Stain Remover/Pre-Wash

Ingredient List—Water, Sodium Methyl 2-Sulfolaurate and Disodium 2-Sulfolaurate, Seed Oil Surfactant, Honeysuckle Extract Composition, Sodium Citrate, Xanthan gum, Protease, Amylase, Mannanase, Pectase, Lipase, Sodium Carbonate
Finished formula pH is in a range of 8.0-10.0

| Trade Name | Weight % | INCI Name | Manufacturer |
|---|---|---|---|
| Water (Deionized) | 58.00-98.94 | Water | |
| Xanthan Gum FF 200 | 0.10-1.00 | Xanthan Gum | Jungbunzlauer, Inc, Newton Centre, |
| Mesh | | | MA |
| Honeysuckle Extract | 0.25-4.00 | Water (and) Ethyl | Greenology Products, LLC, |
| Composition | | Alcohol (and) Lonicera | Raleigh, NC |
| | | Japonica (Honeysuckle) | |
| | | Flower (and) Glycerin | |
| | | (and) Hydrogen | |
| | | Peroxide | |
| TriSodium Citrate | 0.10-2.00 | Sodium Citrate | Jungbunzlauer, Inc, Newton Centre, |
| Dihydrate Fine F6000 | | | MA |
| Ecosurf SA-7 | 0.20-10.00 | Seed Oil Surfactant | Dow Chemical, Midland, MI |
| Alpha-Step PC-48 | 0.20-20.00 | Sodium Methyl 2- | Stepan Company, Northfield, IL |
| | | Sulfolaurate and | |
| | | Disodium 2-Sulfolaurate | |
| Medley Pure 100 L | 0.10-2.00 | Protease, Amylase, | Novozymes North America, Inc, |
| | | Mannanase, Pectase, | |
| | | Lipase | Franklinton, NC |
| Savinase 16 L | 0.10-2.00 | Protease | Novozymes North America, Inc, |
| | | | Franklinton, NC |
| Technical Grade Dense | 0.01-1.00 | Sodium Carbonate | Solvay Chemicals, Inc Princeton, |
| Soda Ash | | | NJ |
| Total | 100.00 | | |

### Preservative Challenge Results

Natural Stain Remover Using < 4.00%
Honeysuckle Extract Composition

| | Inoculum | 0 | Day | Day | Day | Day |
|---|---|---|---|---|---|---|
| Test Organism | Level (CFU/g) | Hours | 7 | 14 | 21 | 28 |
| E. coli | 3.1 × 10⁶ | 2500 | <10 | <10 | <10 | <10 |
| P. aerouginosa | 3.0 × 10⁶ | 2000 | <10 | <10 | <10 | <10 |
| S. aureus | 3.4 × 10⁶ | 1900 | <10 | <10 | <10 | <10 |
| C. albicans | 3.3 × 10⁵ | 2700 | <10 | <10 | <10 | <10 |
| A. (niger) | 5.8 × 10⁵ | 7400 | <10 | <10 | <10 | <10 |
| brasiliensis | | | | | | |

This study also clearly demonstrated that the effectiveness of the composition, in an alkaline pH cleaning composition, against E. coli, P. aeruginosa, S. aureus, A. brasiliensis and C. albicans meets the Category 2 requirement, which is no less than 2.0 log₁₀ reduction from the initial count at 14 days and no increase from the 14-day count at 28 days for bacteria. There was also no increase from the initial calculated count at 14 and 28 days for yeast and mold. For comparison, the same composition was preserved and tested against a known synthetic preservative, benzisothiazolinone, in place of the honeysuckle extract composition, at a level within the manufacturer's recommended dosage range.

Ingredient List—Water, Sodium Methyl 2-Sulfolaurate and Disodium 2-Sulfolaurate, Seed Oil Surfactant, Benzisothiazolinone, Sodium Citrate, Xanthan gum, Protease, Amylase, Mannanase, Pectase, Lipase, Sodium Carbonate
Finished formula pH is in a range of 8.0-10.0

| Trade Name | Weight % | INCI Name | Manufacturer |
|---|---|---|---|
| Water (Deionized) | 61.00-99.09 | Water | |
| Xanthan Gum FF | 0.10-1.00 | Xanthan Gum | Jungbunzlauer, Inc, |
| 200 Mesh | | | Newton Centre, MA |
| Proxel AQ | 0.10-1.00 | Benzisothiazolinone | Lonza, Alpharetta, GA |
| TriSodium Citrate | 0.10-2.00 | Sodium Citrate | Jungbunzlauer, Inc, |
| Dihydrate Fine | | | Newton Centre, MA |
| F6000 | | | |
| Ecosurf SA-7 | 0.20-10.00 | Seed Oil Surfactant | Dow Chemical, Midland, |
| | | | MI |
| Alpha-Step PC-48 | 0.20-20.00 | Sodium Methyl 2- | Stepan Company, |
| | | Sulfolaurate and Disodium | Northfield, IL |
| | | 2-Sulfolaurate | |
| Medley Pure 100 L | 0.10-2.00 | Protease, Amylase, | Novozymes North |
| | | Mannanase, Pectase, Lipase | America, Inc., |
| | | | Franklinton, NC |
| Savinase 16 L | 0.10-2.00 | Protease | Novozymes North |
| | | | America, Inc., |
| | | | Franklinton, NC |
| Technical Grade | 0.01-1.00 | Sodium Carbonate | Solvay Chemicals, Inc |
| Dense Soda Ash | | | Princeton, NJ |
| Total | 100.00 | | |

### Preservative Challenge Results

Natural Stain Remover Using < 1.00% BIT

| | Inoculum | 0 | Day | Day | Day | Day |
|---|---|---|---|---|---|---|
| Test Organism | Level (CFU/g) | Hours | 7 | 14 | 21 | 28 |
| E. coli | 3.7 × 10⁶ | 2000 | <10 | <10 | <10 | <10 |
| P. aerouginosa | 3.1 × 10⁶ | 2700 | <10 | <10 | <10 | <10 |
| S. aureus | 2.6 × 10⁶ | 2300 | <10 | <10 | <10 | <10 |
| C. albicans | 3.3 × 10⁵ | 2100 | <10 | <10 | <10 | <10 |
| A. (niger) | 1.0 × 10⁶ | 12000 | 70 | <10 | <10 | <10 |
| brasiliensis | | | | | | |

### Example 3c

### USDA Certified Organic Free and Clear Laundry Detergent with Honeysuckle Extract Composition

Ingredient List—Water, Sapindus Mukorossi (Organic Soap Berry) Extract, Potassium Cocoate, Honeysuckle Extract Composition, Xanthan Gum, Sodium Carbonate, Dead Sea Salt, Organic Glycerin, Aloe Barbadensis Leaf Juice, Organic Gum Acacia, Organic Guar Gum, Sodium Bicarbonate
Finished formula pH is alkaline, in a range from 9.0-11.0.

| Trade Name | Weight % | INCI Name | Manufacturer |
|---|---|---|---|
| Water (Deionized) | 47.00-98.48 | Water | |
| Dead Sea Salt - | 0.02-2.00 | Dead Sea Salt | Jedwards International, Inc., |
| Edible Fine | | | Braintree, MA |
| Xanthan Gum FF | 0.10-2.00 | Xanthan Gum | Jungbunzlauer, Inc, Newton |
| 200 Mesh | | | Centre, MA |
| TICorganic Aragum | 0.01-1.00 | Organic Gum Acacia, | Tic Gums, Inc., White |
| SDF | | Organic Guar Gum | Marsh, MD |
| Technical Grade | 0.01-2.00 | Sodium Carbonate | Solvay Chemicals, Inc |
| Dense Soda Ash | | | Princeton, NJ |
| Sodium Bicarbonate | 0.01-5.00 | Sodium Bicarbonate | Church & Dwight Co. Inc., |
| Industrial | | | Ewing Township, NJ |
| Terra-Pure Certified | 0.01-2.00 | Aloe Barbadensis Leaf Juice | Terry Laboratories LLC, |
| Spray Dried Aloe | | | Melbourne, FL |
| Vera 200X Powder | | | |
| (TN001CRPLUS) | | | |
| Organic Glycerin | 0.01-5.00 | Organic Glycerin | New Directions Aromatics, |
| | | | Mississauga, ON, Canada |
| Soap Berry Extract | 1.00-20.00 | Sapindus Mukorossi | Vanguard Soap, LLC, |
| | | (Organic Soap Berry) | Memphis, TN |
| | | Extract | |
| Valpro 400-Organic | 0.10-10.00 | Potassium Cocoate | Vanguard Soap, LLC, |
| | | | Memphis, TN |
| Honeysuckle Extract | 0.25-4.00 | Water (and) Ethyl Alcohol | Greenology Products, |
| Composition | | (and) Lonicera Japonica | LLC, Raleigh, NC |
| | | (Honeysuckle) Flower | |
| | | (and) Glycerin (and) | |
| | | Hydrogen Peroxide | |
| Total | 100.00 | | |

### Preservative Challenge Results

Organic Laundry Detergent Using Honeysuckle Extract
Composition Made Fresh

| | Inoculum Level | 0 | Day | Day | Day | Day |
|---|---|---|---|---|---|---|
| Test Organism | (CFU/g) | Hours | 7 | 14 | 21 | 28 |
| Unidentified Gram | 2.45 × 10⁵ | N/A | <10 | <10 | N/A | <10 |
| (+) Coccus | | | | | | |
| Unidentified Gram | 2.80 × 10⁵ | N/A | <10 | <10 | N/A | <10 |
| (+) Bacillus | | | | | | |
| Unidentified Gram | 4.38 × 10⁵ | N/A | <10 | <10 | N/A | <10 |
| (−) Rod | | | | | | |

### Preservative Challenge Results

Organic Laundry Detergent Using Honeysuckle Extract Composition
Aged 12 Weeks at 5° C., 25° C., and 45° C.

| | Inoculum | 0 | Day | Day | Day | Day |
|---|---|---|---|---|---|---|
| Test Organism | Level (CFU/g) | Hours | 7 | 14 | 21 | 28 |
| Unidentified Gram | 2.45 × 10⁵ | N/A | <10 | <10 | N/A | <10 |
| (+) Coccus | | | | | | |
| Unidentified Gram | 2.80 × 10⁵ | N/A | <10 | <10 | N/A | <10 |
| (+) Bacillus | | | | | | |
| Unidentified Gram | 4.38 × 10⁵ | N/A | <10 | <10 | N/A | <10 |
| (−) Rod | | | | | | |

This study demonstrated that the effectiveness of this composition, in a certified organic, alkaline detergent formulation against three unidentified bacteria from different classifications meets the Category 2 requirement, which is no less than 2.0 log₁₀ reduction from the initial count at 14 days and no increase from the 14-day count at 28 days for bacteria. It was also found that the composition was able to effectively preserve against these bacterial strains both fresh and stored at 5° C., 25° C., and 45° C. for 12 weeks before addition to the laundry formulation.

### Example 4

### Antioxidant Characteristics: Antioxidant Capacity and Total Phenolic Content

The extract composition of the present invention was examined for Antioxidant Capacity (AC) and Total Phenolic Content (TPC). The extract composition was evaluated as formulated and undiluted (Table 2), and also formulated and undiluted, without the addition of glycerin (Table 2, with glycerin weight percent dropped to zero and water added to compensate). In addition, the extract composition was further evaluated against an undiluted known antioxidant composition, namely Grapefruit Seed Extract 16006, available from New Directions Aromatics in Mississauga, Ontario, Canada, and is a mixture of grapefruit seed extract, vitamin C and glycerin, as listed in Table 6.

TABLE 6
Ingredient Proportions of Grapefruit Seed Extract

| | Proportions | |
|---|---|---|
| Ingredient | (% w/w) | CAS Number |
| Grapefruit Seed Extract | 40-46 | RN 90045-43-5 |
| Vitamin C | 15-20 | 50-81-7 |
| Glycerin USP | 35-40 | 56-81-5 |
| Moisture | <2.0 | |
| Total | 100.00 | |

### Test Methodology:

The compositions were evaluated using common in vitro methods for antioxidant activity by DPPH. (1,1-dophenyl-2-picrylhydrazyl) free radical scavenging assay and also by ORAC (specific to hydroxyl radical scavenging) assay, along with measuring the total phenolic content by Folin-Ciocalteau assay.

The antioxidant testing was performed using Trolox (a water-soluble analog of Vitamin E) as a standard to determine the Trolox Equivalent (TE). The DPPH. or ORAC value is then calculated from the Trolox Equivalent and expressed as units of value.

DPPH. (1.1-dophenyl-2-picrylhydrazyl) Free Radical Scavenging Assay

DPPH. is one of a few stable and commercially available organic nitrogen radicals and has a UV-vis absorption maximum at 515-517 nm. The molecule DPPH. (1,1-diphenyl-2-picrylhydrazyl) is characterized as a stable free radical by virtue of the delocalization of the spare electron over the molecule as a whole, so that the molecule does not dimerize, as would be the case with most other free radicals. The delocalization of electron gives rise to a deep violet color, characterized by an absorption band in alcohol solution centered at about 517 nm. When a solution of DPPH is mixed with that of a substrate that can donate a hydrogen atom, then this gives rise to the reduced form with the loss of this violet color. In order to evaluate the antioxidant potential through free radical scavenging by the test samples, the change in optical density of DPPH radicals is monitored. Upon reduction, the solution color fades. Testing is conducted using a microplate reader with spectrophotometric detector and 96 well plates. The reaction progress is monitored by a spectrophotometer. DPPH. is utilized in antioxidant research due to its simple reaction system, which involves only the direct reaction(s) between the radical and the antioxidant(s), and have no other interference such as enzyme inhibition or the presence of multiple radicals. The assay was performed in methanol. Results, listed in Table 7, are expressed as micromole Trolox equivalency of a tested material.

### ORAC Method for Hydroxyl Radical Scavenging

When a free-radical generator such as an azo-initiator compound is added to a fluorescent molecule such as beta-phicoerythrin or fluorescein and heated, the azo-initiator produces peroxyl free radicals, which damage the fluorescent molecule, resulting in the loss of fluorescence. Curves of fluorescence intensity versus time are recorded, and the area under the curves with and without addition of an antioxidant is calculated and compared to a standard curve generated using the antioxidant (±)-6-hydroxy-2,5,7,8-tetramethylchromane-2-carboxylic acid, a water-soluble vitamin E analog trademarked by Hoffman LaRoche as Trolox™. Results of this test, as listed in Table 7, are expressed as micromole Trolox equivalency of a tested material.

### Total Phenolics (TPC)

The total phenolic content (TPC) was determined via spectrophotometric method, namely the Folin-Ciocalteau test. In brief, a 1 ml of sample (1 mg/mi) was mixed with 1 ml of Folin-Ciocalteau's phenol reagent. After 5 min, 10 ml of a 7% Na₂C₃ solution was added to the mixture followed by the addition of 13 ml of deionized distilled water and mixed thoroughly. The mixture was kept in the dark for 90 min at 23° C., after which the absorbance was read at 750 nm. The TPC was determined from extrapolation of a calibration curve which was made by preparing gallic acid solution. The estimation of the phenolic compounds was carried out in triplicate. The TPC was expressed as milligrams of gallic acid equivalents (GAE) per g of sample. The antioxidant capacity and total phenolic content values are reported in Table 7.

TABLE 7
Antioxidant Capacity and Total Phenolic Content of Various Extracts

| | | | Antioxidant | |
|---|---|---|---|---|
| | | | Capacity, free | |
| | | Total | radical scavenging | |
| | | Phenolic | | ORAC - |
| | | Content, | DPPH• | Hydroxyl |
| | Table | TPC (mg | (μmole | Radical (μmole |
| Sample | # | GAE/g)¹ | TE/g)² | TE/g)² |
| Grapefruit Seed Extract | 6 | 37.84 | 379.26 | 147.91 |
| in Glycerin | | | | |
| Honeysuckle Extract | 2 | 0.21 | 0.41 | 12.00 |
| Composition | | | | |
| (No Glycerin) | | | | |
| Honeysuckle Extract | 2 | 0.20 | 0.40 | 14.13 |
| Composition | | | | |
| (5% Glycerin) | | | | |

¹milligram gallic acid equivalency per gram
²micromole Trolox equivalency (μmole TE) per gram

The total phenolic content, TPC, reported in Table 7 is in line with the HPLC-UV analytical data we reported for total chlorogenic acid and derivatives (approx. 0.20 mg/g) in Table 3 for honeysuckle extract composition prepared in the same manner. The trend in antioxidant activity of the samples was similar to that of their total polyphenol content, indicating a correlation between the total polyphenol content and antioxidant activity. The results demonstrate total phenolic content is relevant to antioxidant properties and that free radical scavenging activity is present in the extract composition of the present invention. It is believed that glycerin may contribute a slightly larger role in hydroxyl radical scavenging, as shown in the ORAC results of Table 7.

### Example 5

### Sanitizing Capacity

According to the Environmental Protection Agency's (EPA's) Product Performance Test Guidelines (OCSPP 810.2300: Sanitizers for Use on Hard Surfaces—Efficacy Data Recommendations), the product should reduce the number of test microorganisms on a treated surface over those of an untreated control surface (when used as directed). Specifically, three different batches of the formulation containing the extract composition at the tested dosage (one at least 60 days old) are able to show ≥99.9% reduction (a 3-log₁₀ reduction) of the test organisms within 5 minutes on each representative test surface (glass slides in the case of claims for hard, nonporous surfaces), using 5 test carriers and 3 control carriers. The accepted method to determine this is ASTM E-1153¹ for spray products and, therefore, includes the test organisms Staphylococcus aureus American Type Culture Collection (ATCC 6538) and Klebsiella pneumoniae (ATCC 4352), where Klebsiella aerogenes (ATCC 13048) may be substituted for K. pneumoniae. The ASTM method states, “an average of at least 7.5×10⁵ organisms must have survived the inoculated control squares for the test to be valid.”

The sanitizing organic cleaner composition described in Example 5a was tested for efficacy against the test criteria outlined above. In initial screening tests, the results demonstrated that the sanitizing organic cleaner composition is effective and meets the criteria for killing 99.9% of germs on hard, non-porous surfaces within 5 minutes. The data are summarized in Table 8. The composition of Example 5a can further be utilized as a rinse-free antibacterial hard surface soap.

### Example 5a

### Sanitizing Organic Multi-Purpose Cleaner

| Trade Name | Weight % | INCI Name | Manufacturer |
|---|---|---|---|
| Water | 51.00-93.52 | Water | |
| Soap Berry Extract | 0.10-20.00 | Sapindus Mukrossi | Elysian Essentials, LLC, Park |
| | | (Organic Soap Berry) | City UT |
| | | Extract | |
| Organic Ethyl | 1.00-10.00 | Organic Ethyl Alcohol | Florida Distillers Company, Lake |
| Alcohol | | | Alfred, FL |
| Coladet OKC-40 | 0.50-10.00 | Potassium Cocoate | Colonial Chemical, Inc., South |
| | | | Pittsburg, TN |
| Salt Purex TFC | 0.10-1.00 | Sodium Chloride | Morton Salt, Inc., Chicago, IL |
| Common Fine | | | |
| FCC | | | |
| Technical Grade | 0.01-2.00 | Sodium Carbonate | Tronox, Stamford, CT |
| Dense Soda Ash | | | |
| Fragrance | 0.02-2.00 | Fragrance | |
| (optional) | | | |
| Honeysuckle | 0.25-4.00 | Water (and) Ethyl | Greenology Products, LLC, |
| Extract | | Alcohol (and) | Raleigh, NC |
| Composition | | Lonicera Japonica | |
| | | (Honeysuckle) Flower | |
| | | (and) Glycerin (and) | |
| | | Hydrogen Peroxide | |
| Total | 100.00 | | |

The cleaner product composition of Example 5a was dispensed via a bottle equipped with a trigger sprayer, for a total of 3 sprays, at approximately 1.3 cc per spray, at a distance of 4-6 inches from the substrate. Table 8 summarizes test results for carriers treated with the cleaner of Example 5a, at a total delivery of approximately 4 cc.

TABLE 8
Non-GLP Sanitizing Performance of Example 5, Organic Multi-Purpose Cleaner
with Honeysuckle Extract Composition

| | | Weight % | | | | Geometric | |
|---|---|---|---|---|---|---|---|
| | Contact | Extract | | Replicate | | Mean | Percent |
| Test Micro- | Time, | Composition in | Carrier | (CFU/ | Log₁₀ | (CFU/ | Reduction |
| organism | Minutes | Example 5a | Replicate | Carrier) | Density | Carrier) | (%) |
| Klebsiella | 5 | 1.5 | 1 | <=1.00 × | <=1.00 | <= 1.00 × | >=99.9998 |
| aerogenes | | | | 10¹ | | 10¹ | |
| ATCC | 5 | 1.5 | 2 | <=1.00 × | <=1.00 | | |
| 13048 | | | | 10¹ | | | |
| | 5 | 1.5 | 3 | <=1.00 × | <=1.00 | | |
| | | | | 10¹ | | | |
| | 5 | 1.5 | 4 | <=1.00 × | <=1.00 | | |
| | | | | 10¹ | | | |
| | 5 | 1.5 | 5 | <=1.00 × | <=1.00 | | |
| | | | | 10¹ | | | |
| Staphylo- | 5 | 1.0 | 1 | 1.25 × 10³ | 3.10 | 1.64 × 10³ | 99.90 |
| coccus | 5 | 1.0 | 2 | 1.09 × 10³ | 3.04 | | |
| aureus | 5 | 1.0 | 3 | 2.02 × 10³ | 3.31 | | |
| ATCC | 5 | 1.0 | 4 | 2.71 × 10³ | 3.43 | | |
| 6538 | 5 | 1.0 | 5 | 1.57 × 10³ | 3.20 | | |

With the results of Table 8, the extract composition can also be used as the active ingredient in an antibacterial liquid hand dish soap when present at a dosage of at least between 0.25% and 4.00%, as described in Example 5b.

### Example 5b

### Sulfate-Free Liquid Hand Dish Detergent Soap

| Trade Name | Weight % | INCI Name | Manufacturer |
|---|---|---|---|
| Water | 40.00-95.32 | Water | |
| Alpha Olefin | 3.00-30.00 | Sodium C14-16 | Colonial Chemical, Inc., South |
| Sulfonate | | Olefin Sulfonate | Pittsburg, TN |
| Mirataine BET C30 | 1.00-10.00 | Cocamidopropyl | Solvay Canada Inc., Toronto, ON |
| | | Betaine | |
| Ninol CAA | 0.20-2.00 | Dodecanamide, N, | Stepan Company, Northfield, IL |
| | | N-dimethyl and | |
| | | Tetradecanamide, N, | |
| | | N-dimethyl | |
| Salt Purex TFC | 0.10-5.00 | Sodium Chloride | Morton Salt, Inc., Chicago, IL |
| Common Fine FCC | | | |
| Sodium Citrate | 0.10-5.00 | Sodium Citrate | Jungbunzlauer Inc., Newton, MA |
| Citric Acid | 0.01-2.00 | Citric Acid | Jungbunzlauer Inc., Newton, MA |
| Honeysuckle Extract | 0.25-4.00 | Water (and) Ethyl | Greenology Products, LLC, |
| Composition | | Alcohol (and) | Raleigh, NC |
| | | Lonicera Japonica | |
| | | (Honeysuckle) | |
| | | Flower (and) | |
| | | Glycerin (and) | |
| | | Hydrogen Peroxide | |
| Fragrance (optional) | 0.02-2.00 | Fragrance | |
| Total | 100.00 | | |

### Example 6

### Disinfecting Capacity

The extract composition of the present invention may have general or broad spectrum disinfectant efficacy when used at a dosage of 0.25% or above, for example, in the Organic Multi-Purpose Cleaner composition of Example 5a.

As discussed above, disinfectants are capable of destroying, neutralizing and/or inhibiting the growth of disease-carrying microorganisms and may also be used in the air, on hard, inanimate surfaces and/or objects. They are capable of destroying or irreversibly inactivating fungi and bacteria but not necessarily their spores. Generally, a broad-spectrum disinfectant possesses efficacy against both gram-negative and gram-positive bacteria. According to some standards, the product should achieve total kill, at a log 6 reduction of organisms, at double the contact time required for sanitizing (e.g., 10 minutes versus 5 minutes). According to other requirements, the product is tested against Staphylococcus aureus (ATCC 6538) and Salmonella enterica (ATCC 10708), where Pseudomonas aeruginosa (ATCC 15442) may be used instead of S. enterica.

In some cases, the extract compositions of the present invention may be tested against one or more specific target bacteria other than the generally used test microorganisms discussed above. Such testing may involve testing, for example, ten carriers against each of two batches of the product at or below the nominal concentration. The evaluation of success in this case is that the product should kill all of the test microorganisms on all ten carriers (i.e., no positive carriers).

Virucidal capacity of the compositions of the present invention for use on hard, non-porous surfaces may be tested by inoculating the specific virus to be treated (or EPA acceptable surrogate) onto hard, non-porous surfaces (for example, glass carriers or Petri dishes), allowed to dry, and then treated with the product according to the desired usage. Evaluation of a reduction on each surface in the presence or absence of cytotoxicity will be determined.

### Example 7

### Additional Products Formulations

The following additional product Examples were also tested for product preservation efficacy per the Preservation Challenge Testing as described in Example 2, with passing results.

### Example 7a

### Organic Laundry Detergent

| Trade Name | Weight % | INCI Name | Manufacturer |
|---|---|---|---|
| Water | 0.00-46.32 | Water | |
| Xanthan Gum FF | 0.20-2.00 | Xanthan Gum | Jungbunzlauer, Inc, Newton, |
| 200 Mesh | | | MA |
| Soap Berry Extract | 50.00-80.00 | Sapindus Mukrossi | Elysian Essentials, LLC, Park |
| | | (Organic Soap Berry) | City UT |
| | | Extract | |
| Coladet OKC-40 | 2.00-20.00 | Potassium Cocoate | Colonial Chemical, Inc., South |
| | | | Pittsburg, TN |
| Salt Purex TFC | 0.10-1.00 | Sodium Chloride | Morton Salt, Inc., Chicago, IL |
| Common Fine FCC | | | |
| Technical Grade | 0.01-2.00 | Sodium Carbonate | Tronox, Stamford, CT |
| Dense Soda Ash | | | |
| Sodium Citrate | 0.20-5.00 | Sodium Citrate | Jungbunzlauer, Inc, Newton, |
| | | | MA |
| Honeysuckle | 0.25-4.00 | Water (and) Ethyl | Greenology Products, LLC, |
| Extract | | Alcohol (and) Lonicera | Raleigh, NC |
| Composition | | Japonica | |
| | | (Honeysuckle) Flower | |
| | | (and) Glycerin (and) | |
| | | Hydrogen Peroxide | |
| Fragrance | 0.02-2.00 | Fragrance | |
| (optional) | | | |
| Total | 100.00 | | |

### Example 7b

### Organic Hand Dishwashing Soap

| Trade Name | Weight % | INCI Name | Manufacturer |
|---|---|---|---|
| Water | 21.00-87.75 | Water | |
| Soap Berry | 10.00-50.00 | Sapindus Mukrossi | Elysian Essentials, LLC, Park City |
| Extract | | (Organic Soap Berry) | UT |
| | | Extract | |
| Coladet OKC-40 | 2.00-25.00 | Potassium Cocoate | Colonial Chemical, Inc., South |
| | | | Pittsburg, TN |
| Honeysuckle | 0.25-4.00 | Water (and) Ethyl | Greenology Products, LLC, |
| Extract | | Alcohol (and) | Raleigh, NC |
| Composition | | Lonicera Japonica | |
| | | (Honeysuckle) Flower | |
| | | (and) Glycerin (and) | |
| | | Hydrogen Peroxide | |
| Total | 100.00 | | |

### Example 7c

### Organic Fruit and Vegetable Wash

| Trade Name | Weight % | INCI Name | Manufacturer |
|---|---|---|---|
| Water | 52.50-98.03 | Water | |
| Soap Berry Extract | 0.10-20.00 | Sapindus Mukrossi | Elysian Essentials, LLC, Park |
| | | (Organic Soap Berry) | City UT |
| | | Extract | |
| Organic Ethyl | 1.00-10.00 | Organic Ethyl Alcohol | Florida Distillers Company, Lake |
| Alcohol | | | Alfred, FL |
| Coladet OKC-40 | 0.50-10.00 | Potassium Cocoate | Colonial Chemical, Inc., South |
| | | | Pittsburg, TN |
| Salt Purex TFC | 0.10-1.00 | Sodium Chloride | Morton Salt, Inc., Chicago, IL |
| Common Fine FCC | | | |
| Technical Grade | 0.01-2.00 | Sodium Carbonate | Tronox, Stamford, CT |
| Dense Soda Ash | | | |
| Organic Lemon | 0.01-0.50 | Citrus Limon (Lemon) | New Directions Aromatics Inc., |
| Essential Oil | | Peel Oil | Mississauga, Ontario |
| Honeysuckle | 0.25-4.00 | Water (and) Ethyl | Greenology Products, LLC, |
| Extract | | Alcohol (and) | Raleigh, NC |
| Composition | | Lonicera Japonica | |
| | | (Honeysuckle) Flower | |
| | | (and) Glycerin (and) | |
| | | Hydrogen Peroxide | |
| Total | 100.00 | | |

### Example 7d

### Sulfate-Free Hand Soap

| Trade Name | Weight % | INCI Name | Manufacturer |
|---|---|---|---|
| Water | 37.00-95.32 | Water | |
| Alpha Olefin | 3.00-30.00 | Sodium C14-16 | Colonial Chemical, Inc., South |
| Sulfonate | | Olefin Sulfonate | Pittsburg, TN |
| Mirataine BET C30 | 0.10-10.00 | Cocamidopropyl | Solvay Canada Inc., Toronto, ON |
| | | Betaine | |
| Glycerin | 1.00-8.00 | Glycerin | New Directions Aromatics Inc., |
| | | | Mississauga, Ontario |
| Ninol CAA | 0.20-2.00 | Dodecanamide, N, | Stepan Company, Northfield, IL |
| | | N-dimethyl and | |
| | | Tetradecanamide, N, | |
| | | N-dimethyl | |
| Salt Purex TFC | 0.10-5.00 | Sodium Chloride | Morton Salt, Inc., Chicago, IL |
| Common Fine FCC | | | |
| Citric Acid | 0.01-2.00 | Citric Acid | Jungbunzlauer Inc., Newton, MA |
| Honeysuckle | 0.25-4.00 | Water (and) Ethyl | Greenology Products, LLC, |
| Extract | | Alcohol (and) | Raleigh, NC |
| Composition | | Lonicera Japonica | |
| | | (Honeysuckle) | |
| | | Flower (and) | |
| | | Glycerin (and) | |
| | | Hydrogen Peroxide | |
| Fragrance | 0.02-2.00 | Fragrance | |
| (optional) | | | |
| Total | 100.00 | | |

### Example 7e

### Sulfate-Free Body Wash

| Trade Name | Weight % | INCI Name | Manufacturer |
|---|---|---|---|
| Water | 37.00-95.32 | Water | |
| Alpha Olefin | 3.00-30.00 | Sodium C14-16 | Colonial Chemical, Inc., South |
| Sulfonate | | Olefin Sulfonate | Pittsburg, TN |
| Mirataine BET | 0.10-10.00 | Cocamidopropyl | Solvay Canada Inc., Toronto, ON |
| C30 | | Betaine | |
| Ninol CAA | 0.20-2.00 | Dodecanamide, N, N- | Stepan Company, Northfield, IL |
| | | dimethyl and | |
| | | Tetradecanamide, N, | |
| | | N-dimethyl | |
| Salt Purex TFC | 0.10-5.00 | Sodium Chloride | Morton Salt, Inc., Chicago, IL |
| Common Fine | | | |
| FCC | | | |
| Glycerin | 1.00-8.00 | Glycerin | New Directions Aromatics Inc., |
| | | | Mississauga, Ontario |
| Citric Acid | 0.01-2.00 | Citric Acid | Jungbunzlauer Inc., Newton, MA |
| Honeysuckle | 0.25-4.00 | Water (and) Ethyl | Greenology Products, LLC, |
| Extract | | Alcohol (and) | Raleigh, NC |
| Composition | | Lonicera Japonica | |
| | | (Honeysuckle) | |
| | | Flower (and) | |
| | | Glycerin (and) | |
| | | Hydrogen Peroxide | |
| Fragrance | 0.02-2.00 | Fragrance | |
| (optional) | | | |
| Total | 100.00 | | |

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A composition comprising a mixture of at least one plant infusion or extract and hydrogen peroxide.

2. The composition of claim 1, wherein the at least one plant infusion or extract is derived from a plant selected from the group consisting of honeysuckle, artichoke, sunflower, rose, lilac, lavender, wisteria, yarrow, chamomile, guava, peppermint, lemongrass, garlic, clove, ginger, geranium, cinnamon, rosemary, thyme, oregano, tarragon, green coffee bean (Coffea arabica), juniper, nutmeg, anise, noni, ginger, guava, pomegranate, sage, jambolan, Cremaspora, St. John's Wort (Hypericum), parsley (Heteromorpha), cheesewoods (Pittosporum), Bolusanthus, Calpurnia, Maesa, Elaeodendron, African mulberry and combinations thereof.

3. The composition of claim 1 or 2, wherein the plant is a member of the genus Lonicera.

4. The composition according to any of the preceding claims, wherein the plant is a member of the species Lonicera caprifolium (goat-leaf honeysuckle, perfoliate honeysuckle), Lonicera periclymenum (European honeysuckle or woodbine), Lonicera japonica (Japanese honeysuckle, white honeysuckle, or Chinese honeysuckle), Lonicera sempervirens (coral honeysuckle or trumpet honeysuckle), Lonicera hildebrandiana (Burmese honeysuckle), Lonicera tartarica (Tartarian honeysuckle), Lonicera fragrantissima (winter honeysuckle), Lonicera pileate (privet honeysuckle), Lonicera nitida (box honeysuckle) and Lonicera syringantha (lilac-flowered honeysuckle).

5. The composition according to any of the preceding claims, wherein the at least one plant infusion or extract is derived from a honeysuckle plant.

6. The composition according to any of the preceding claims, wherein the at least one plant infusion or extract is derived from Lonicera japonica (Japanese honeysuckle).

7. The composition according to any of the preceding claims, wherein the at least one plant infusion or extract is derived from a bud, leaf and/or stem, blossom and/or fruit of the plant.

8. The composition according to any of the preceding claims, wherein the composition further comprises glycerin.

9. The composition according to any of the preceding claims, wherein the at least one plant infusion or extract comprises a plant material derived from a flower bud and a solvent selected from the group consisting of water, alcohol, chloroform, acetone, petroleum ether, an ionic liquid and combinations thereof.

10. The composition according to any of the preceding claims, wherein the at least one plant infusion or extract comprises 0.000 to 99.999% water, 0.000 to 95.000% alcohol and 0.001 to 99.999% honeysuckle flower buds.

11. The composition according to any of the preceding claims, wherein the at least one plant infusion or extract further comprises 0.001% to 99.999% polyphenols and/or phenolic compounds.

12. The composition of claim 11, wherein the at least one plant infusion or extract further comprises an organic acid, a flavonoid or combinations thereof.

13. The composition according to any of the preceding claims, wherein the composition is a sulfate free, synthetic, natural, or organic composition, or combinations thereof.

14. The composition according to any of the preceding claims, wherein the composition is a certified organic composition.

15. The composition according to any of the preceding claims, wherein the at least one plant infusion or extract is a synthetic, natural or organic infusion or extract.

16. The composition according to any of the preceding claims, wherein the at least one plant infusion or extract is a certified organic infusion or extract.

17. The composition according to any of the preceding claims, wherein the composition has mitigating and/or preservation properties.

18. The composition according to any of the preceding claims, wherein the composition is substantially free of parabens or paraben free.

19. A composition comprising a phenolic or polyphenol compound and hydrogen peroxide.

20. The composition of claim 19, wherein the phenolic or polyphenol compound is an organic acid, a flavonoid or combinations thereof.

21. The composition of claim 20, wherein the organic acid is a hydroxycinnamic acid and/or its derivatives.

22. The composition of claim 21, wherein the hydroxycinnamic acid is chlorogenic acid, isochlorogenic acid, caffeic acid, hexadecanoic acid, myristic acid or combinations thereof.

23. The composition of any one of claims 19-22, wherein the composition is substantially free of parabens or paraben free.

24. A plant infusion or extract comprising:
(a) a solvent selected from the group consisting of water, alcohol, chloroform, acetone, petroleum ether, an ionic liquid and combinations thereof,
(b) hydrogen peroxide; and
(c) a plant material.

25. The plant infusion or extract of claim 24, wherein the plant is a member of the species Lonicera caprifolium (goat-leaf honeysuckle, perfoliate honeysuckle), Lonicera periclymenum (European honeysuckle or Woodbine), Lonicera japonica (Japanese honeysuckle, white honeysuckle, or Chinese honeysuckle), Lonicera sempervirens (coral honeysuckle or trumpet honeysuckle), Lonicera hildebrandiana (Burmese honeysuckle), Lonicera tartarica (Tartarian honeysuckle), Lonicera fragrantissima (winter honeysuckle), Lonicera pileate (privet honeysuckle), Lonicera nitida (box honeysuckle) and Lonicera syringantha (lilac-flowered honeysuckle).

26. The plant infusion or extract of claim 24 or 25, wherein the infusion or extract is formed by adding a whole, chopped and/or ground plant material to water and/or alcohol; filtering the solution resulting from the addition of the whole, chopped and/or ground plant material to the water and/or alcohol; and adding hydrogen peroxide to the filtered solution, and optionally adding glycerin.

27. A product comprising the composition of one of claims 1-23 or the plant infusion or extract of one of claims 24-26.

28. The product of claim 27, wherein the product is a food, beverage, cosmetic, pharmaceutical, nutraceutical, agricultural treatment product, water treatment product, cleaning composition, consumer product or personal care product.

29. The product of claim 27 or 28, wherein the product is a laundry detergent, laundry additive, fabric sanitizer, automatic dishwashing detergent, manual dishwashing detergent, multi-surface cleaner, hard surface cleaner, rinse-free antibacterial hard surface soap, hand soap, rinse-free hand soap, hand sanitizer, shampoo or animal cleaning product.

30. The product of one of claims 27-29, wherein the product is a laundry detergent, a laundry additive, an automatic dishwashing detergent or manual dishwashing detergent.

31. The product of any one of claims 27-29, wherein the product is an animal care and/or cleaning product selected from the group consisting of shampoo, cage cleaner, cleaning wipes and deodorizing spray.

32. The product of any one of claims 27-31, wherein the product is a sulfate free, natural, organic or certified organic product.

33. The product of any one of claims 27-32, wherein the product is an antioxidant, antibacterial, preservative, disinfectant and/or sanitizer.

34. A method of making a plant infusion or extract, the method comprising:
(a) adding a whole, chopped and/or ground plant material to water and/or alcohol;
(b) filtering the solution of (a) to obtain a filtered solution; and
(c) adding hydrogen peroxide to the filtered solution to obtain a plant infusion or extract.

35. The method of claim 34, wherein the method further comprises adding glycerin to the solution provided in (c).

36. The method of claim 34 or 35, wherein the starting materials are synthetic, natural, organic or a combination thereof.

37. A method as in any one of claims 34, 35 and 36, in which the starting materials are at least 95% certified organic.

38. A method as in any one of claims 34, 35 and 36, in which the plant infusion or extract is synthetic.

39. A method as in any one of claims 34, 35, 36 and 37, in which the plant infusion or extract is at least 95% certified organic.

40. The method as in any one of claims 34, 35, 36, 37, 38 and 39, in which the plant infusion or extract is substantially paraben free.

41. The method as in any one of claims 34, 35, 36, 37, 38 and 39, in which the plant infusion or extract is paraben free.
